SEQUENCE LISTING (93 sequences)

SEQ ID NO: 1 - protein, 15 aa, Artificial
  Synthetic Peptide
CFELDGTCFDWAQEC

SEQ ID NO: 2 - protein, 15 aa, Artificial
  Synthetic Peptide
CVNFGSVCWDPDSRC

SEQ ID NO: 3 - protein, 12 aa, Artificial
  Synthetic Peptide
CVPCADFPIWYC

SEQ ID NO: 4 - protein, 12 aa, Artificial
  Synthetic Peptide
CVPCADFPIWWC

SEQ ID NO: 5 - protein, 12 aa, Artificial
  Synthetic Peptide
CTPCADFPIWSC

SEQ ID NO: 6 - protein, 12 aa, Artificial
  Synthetic Peptide
CTPCADFPIHTC

SEQ ID NO: 7 - protein, 12 aa, Artificial
  Synthetic Peptide
CVHCADFPIWGC

SEQ ID NO: 8 - protein, 12 aa, Artificial
  Synthetic Peptide
CVPCADFPIWGC

SEQ ID NO: 9 - protein, 12 aa, Artificial
  Synthetic Peptide
CVMCADFPIWGC

SEQ ID NO: 10 - protein, 12 aa, Artificial
  Synthetic Peptide
CTPCADFPIWYC

SEQ ID NO: 11 - protein, 12 aa, Artificial
  Synthetic Peptide
CTPCADFPILTC

SEQ ID NO: 12 - protein, 12 aa, Artificial
  Synthetic Peptide
CVACADFPIWGC

SEQ ID NO: 13 - protein, 12 aa, Artificial
  Synthetic Peptide
CTPCADFPIYGC

SEQ ID NO: 14 - protein, 12 aa, Artificial
  Synthetic Peptide
CTPCADFPILDC

SEQ ID NO: 15 - protein, 12 aa, Artificial
  Synthetic Peptide
CVKCADFPIWGC

SEQ ID NO: 16 - protein, 12 aa, Artificial
  Synthetic Peptide
CTPCADMPIWTC

SEQ ID NO: 17 - protein, 12 aa, Artificial
  Synthetic Peptide
CIPCADFPIWGC

SEQ ID NO: 18 - protein, 12 aa, Artificial
  Synthetic Peptide
CIPCADFPISVC

SEQ ID NO: 19 - protein, 12 aa, Artificial
  Synthetic Peptide
CVPCADFPISFC

SEQ ID NO: 20 - protein, 12 aa, Artificial
  Synthetic Peptide
CIPCADFPISFC

SEQ ID NO: 21 - protein, 12 aa, Artificial
  Synthetic Peptide
CVPCADFPISVC

SEQ ID NO: 22 - protein, 12 aa, Artificial
  Synthetic Peptide
CVPCADFPIFTC

SEQ ID NO: 23 - protein, 12 aa, Artificial
  Synthetic Peptide
CIPCADFPIFTC

SEQ ID NO: 24 - protein, 12 aa, Artificial
  Synthetic Peptide
CTPCADFPIWGC

SEQ ID NO: 25 - protein, 13 aa, Artificial
  Synthetic Peptide
CDFTMPCENWKYC

SEQ ID NO: 26 - protein, 13 aa, Artificial
  Synthetic Peptide
CDFTMPCPNWNAC

SEQ ID NO: 27 - protein, 13 aa, Artificial
  Synthetic Peptide
CDFTMPCQMWEQC

SEQ ID NO: 28 - protein, 13 aa, Artificial
  Synthetic Peptide
CIFDYDCDAWSAC

SEQ ID NO: 29 - protein, 15 aa, Artificial
  Synthetic Peptide
CFWLDGECFDWNHEC

SEQ ID NO: 30 - protein, 15 aa, Artificial
  Synthetic Peptide
CFHLDGECFDLENTC

SEQ ID NO: 31 - protein, 9 aa, Artificial
  Synthetic Peptide
CAWLCPNLC

SEQ ID NO: 32 - protein, 15 aa, Artificial
  Synthetic Peptide
CFSLDGECFDLSGEC

SEQ ID NO: 33 - protein, 15 aa, Artificial
  Synthetic Peptide
CFTLDGECFDWTHEC

SEQ ID NO: 34 - protein, 15 aa, Artificial
  Synthetic Peptide
CFKLDGVCFDLFHEC

SEQ ID NO: 35 - protein, 15 aa, Artificial
  Synthetic Peptide
CFMLDGECFDLNKEC

SEQ ID NO: 36 - protein, 15 aa, Artificial
  Synthetic Peptide
CFKLDGECFDWTHEC

SEQ ID NO: 37 - protein, 15 aa, Artificial
  Synthetic Peptide
CFTLDGECFDWDAEC

SEQ ID NO: 38 - protein, 15 aa, Artificial
  Synthetic Peptide
CFELDGSCFDFDHEC

SEQ ID NO: 39 - protein, 15 aa, Artificial
  Synthetic Peptide
CFTLDGECFDVNREC

SEQ ID NO: 40 - protein, 15 aa, Artificial
  Synthetic Peptide
CFWLDHECFDWTHEC

SEQ ID NO: 41 - protein, 15 aa, Artificial
  Synthetic Peptide
CFQLDGECFDIYREC

SEQ ID NO: 42 - protein, 15 aa, Artificial
  Synthetic Peptide
CFELDGNCFDWTHEC

SEQ ID NO: 43 - protein, 15 aa, Artificial
  Synthetic Peptide
CFHLDGECFDYEHEC

SEQ ID NO: 44 - protein, 15 aa, Artificial
  Synthetic Peptide
CFSLDGECFDIASEC

SEQ ID NO: 45 - protein, 15 aa, Artificial
  Synthetic Peptide
CFQLDGECFDTSHEC

SEQ ID NO: 46 - protein, 15 aa, Artificial
  Synthetic Peptide
CFSLDGACFDWTHEC

SEQ ID NO: 47 - protein, 15 aa, Artificial
  Synthetic Peptide
CFVLDGECFDYYEEC

SEQ ID NO: 48 - protein, 15 aa, Artificial
  Synthetic Peptide
CFRLDDECFDWTHEC

SEQ ID NO: 49 - protein, 15 aa, Artificial
  Synthetic Peptide
CFRLDGVCFDLDDEC

SEQ ID NO: 50 - protein, 15 aa, Artificial
  Synthetic Peptide
CFRLDGECFDMGQEC

SEQ ID NO: 51 - protein, 15 aa, Artificial
  Synthetic Peptide
CFTLDGACFDLDGEC

SEQ ID NO: 52 - protein, 15 aa, Artificial
  Synthetic Peptide
CFTLDGQCFDWTHEC

SEQ ID NO: 53 - protein, 15 aa, Artificial
  Synthetic Peptide
CFLLDGECFDWMQEC

SEQ ID NO: 54 - protein, 15 aa, Artificial
  Synthetic Peptide
CFELDGDCFDWTHEC

SEQ ID NO: 55 - protein, 15 aa, Artificial
  Synthetic Peptide
CFTLDGTCFDWTHEC

SEQ ID NO: 56 - protein, 15 aa, Artificial
  Synthetic Peptide
CFHLDGVCFDWTHEC

SEQ ID NO: 57 - protein, 15 aa, Artificial
  Synthetic Peptide
CFYLDGTCFDWTHEC

SEQ ID NO: 58 - protein, 15 aa, Artificial
  Synthetic Peptide
CFLLDGECFDWAQEC

SEQ ID NO: 59 - protein, 15 aa, Artificial
  Synthetic Peptide
CFHLDGECFDLAKTC

SEQ ID NO: 60 - protein, 15 aa, Artificial
  Synthetic Peptide
CFELDGECFHFGEPC

SEQ ID NO: 61 - protein, 15 aa, Artificial
  Synthetic Peptide
CFVLDGECFEIGERC

SEQ ID NO: 62 - protein, 15 aa, Artificial
  Synthetic Peptide
CFTLDGECFDLDGWC

SEQ ID NO: 63 - protein, 15 aa, Artificial
  Synthetic Peptide
CFLLDGECFDLIGEC

SEQ ID NO: 64 - protein, 15 aa, Artificial
  Synthetic Peptide
CFELDGECFSFPGTC

SEQ ID NO: 65 - protein, 15 aa, Artificial
  Synthetic Peptide
CFELDGECFSWPYPC

SEQ ID NO: 66 - protein, 15 aa, Artificial
  Synthetic Peptide
CFTLDGECFLLGENC

SEQ ID NO: 67 - protein, 15 aa, Artificial
  Synthetic Peptide
CFWLDGECFDLGGQC

SEQ ID NO: 68 - protein, 15 aa, Artificial
  Synthetic Peptide
CFELDGECFDLDNQC

SEQ ID NO: 69 - protein, 15 aa, Artificial
  Synthetic Peptide
CFWLDGECFDLYGGC

SEQ ID NO: 70 - protein, 15 aa, Artificial
  Synthetic Peptide
CFRLDGECFDISNEC

SEQ ID NO: 71 - protein, 15 aa, Artificial
  Synthetic Peptide
CFELDGECFNIGSKC

SEQ ID NO: 72 - protein, 14 aa, Artificial
  Synthetic Peptide
CFWLDGECFDFGGC

SEQ ID NO: 73 - protein, 15 aa, Artificial
  Synthetic Peptide
CFTLDGACFDWTHEC

SEQ ID NO: 74 - protein, 12 aa, Artificial
  Synthetic Peptide
CDYCVRLGLTGC

SEQ ID NO: 75 - protein, 12 aa, Artificial
  Synthetic Peptide
CGWCSDQIDGFC

SEQ ID NO: 76 - protein, 12 aa, Artificial
  Synthetic Peptide
CAWCSDPIDGFC

SEQ ID NO: 77 - protein, 12 aa, Artificial
  Synthetic Peptide
CDWCIDPGVSFC

SEQ ID NO: 78 - protein, 12 aa, Artificial
  Synthetic Peptide
CSWCVDDGLPFC

SEQ ID NO: 79 - protein, 12 aa, Artificial
  Synthetic Peptide
CTWCVDDGLSFC

SEQ ID NO: 80 - protein, 12 aa, Artificial
  Synthetic Peptide
CTWCVDDETWNC

SEQ ID NO: 81 - protein, 12 aa, Artificial
  Synthetic Peptide
CDYCIRLGLTGC

SEQ ID NO: 82 - protein, 12 aa, Artificial
  Synthetic Peptide
CDWCTDNIPGIC

SEQ ID NO: 83 - protein, 15 aa, Artificial
  Synthetic Peptide
CIRYGDICYDPDHSC

SEQ ID NO: 84 - protein, 15 aa, Artificial
  Synthetic Peptide
CIRYGDICFHPDYTC

SEQ ID NO: 85 - protein, 15 aa, Artificial
  Synthetic Peptide
CINYANICLDTEKMC

SEQ ID NO: 86 - protein, 9 aa, Artificial
  Synthetic Peptide
CNFLCDDLC

SEQ ID NO: 87 - protein, 15 aa, Artificial
  Synthetic Peptide
  Xaa (3)..(3): Xaa represents any amino acid residue
  Xaa (6)..(7): Xaa represents any amino acid residue
  Xaa (11)..(13): Xaa represents any amino acid residue
  Xaa (14)..(14): Xaa is either absent or represents any amino acid residue
CFXLDXXCFDXXXXC

SEQ ID NO: 88 - protein, 15 aa, Artificial
  Synthetic Peptide
  Xaa (3)..(3): Xaa is R or N
  Xaa (5)..(5): Xaa is G or A
  Xaa (6)..(6): Xaa is D or N
  Xaa (9)..(9): Xaa represents any amino acid residue
  Xaa (10)..(10): Xaa is D or H
  Xaa (11)..(11): Xaa is P or T
  Xaa (12)..(12): Xaa is D or E
  Xaa (13)..(14): Xaa represents any amino acid residue
CIXYXXICXXXXXXC

SEQ ID NO: 89 - protein, 15 aa, Artificial
  Synthetic Peptide
  Xaa (3)..(3): Xaa represents any amino acid residue
  Xaa (10)..(11): Xaa represents any amino acid residue
  Xaa (12)..(12): Xaa is G or P
  Xaa (13)..(14): Xaa represents any amino acid residue
CFXLDGECFXXXXXC

SEQ ID NO: 90 - protein, 12 aa, Artificial
  Synthetic Peptide
  Xaa (2)..(3): Xaa represents any amino acid residue
  Xaa (7)..(7): Xaa is F or M
  Xaa (10)..(11): Xaa represents any amino acid residue
CXXCADXPIXXC

SEQ ID NO: 91 - protein, 9 aa, Artificial
  Synthetic Peptide
  Xaa (2)..(2): Xaa is A or N
  Xaa (3)..(3): Xaa is W or F
  Xaa (6)..(6): Xaa is P or D
  Xaa (7)..(7): Xaa is N or D
CXXLCXXLC

SEQ ID NO: 92 - protein, 13 aa, Artificial
  Synthetic Peptide
  Xaa (8)..(9): Xaa represents any amino acid residue
  Xaa (11)..(12): Xaa represents any amino acid residue
CDFTMPCXXWXXC

SEQ ID NO: 93 - protein, 14 aa, Artificial
  Synthetic Peptide
ACTPCADFPIWGCA

(19) United States
(12) Patent Application Publication
(10) Pub. No.: US 2022/0133733 A1
(43) Pub. Date: May 5, 2022

(54) BICYCLIC PEPTIDE LIGANDS SPECIFIC FOR CD38

(71) Applicant: BicycleRD Limited, Cambridge (GB)

(72) Inventors: Leonardo BALDASSARRE, Cambridge (GB); Rachid LANI, Cambridge (GB); Silvia PAVAN, Cambridge (GB); Catherine STACE, Cambridge (GB); Daniel TEUFEL, Cambridge (GB)

(21) Appl. No.: 17/422,943

(22) PCT Filed: Jan. 15, 2020
(86) PCT No.: PCT/GB2020/050074

§ 371 Date: Jul. 14, 2021

(30) Foreign Application Priority Data
Jan. 15, 2019 (GB) 1900530.5

(51) Int. Cl. A61K 31/537; A61K 47/64; A61P 35/00

(52) U.S. Cl. CPC A61K 31/537; A61P 35/00; A61K 47/64

(57) ABSTRACT

The present invention relates to polypeptides which are covalently bound to aromatic molecular scaffolds such that two or more peptide loops are subtended between attachment points to the scaffold. In particular, the invention describes peptides which are high affinity binders of CD38. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by CD38.

### FIELD OF THE INVENTION

The present invention relates to polypeptides which are covalently bound to aromatic molecular scaffolds such that two or more peptide loops are subtended between attachment points to the scaffold. In particular, the invention describes peptides which are high affinity binders of CD38. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by CD38.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin aVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a peptide ligand specific for CD38 comprising a polypeptide comprising at least three cysteine residues, separated by at least two loop sequences, and an aromatic molecular scaffold which forms covalent bonds with the cysteine residues of the polypeptide such that at least two polypeptide loops are formed on the molecular scaffold.

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by CD38.

### BRIEF DESCRIPTION OF THE FIGURES

FIG. 1: Body weight changes after administering BT66BDC1 to female Balb/c nude mice bearing HT1080. Data points represent group mean body weight. Error bars represent standard error of the mean (SEM).

FIG. 2: Tumor volume trace after administering BT66BDC1 to female Balb/c nude mice bearing HT1080 xenograft. Data points represent group mean, error bars represent standard error of the mean (SEM).

FIG. 3: Body weight changes after administering BT66BDC1 to female CB17-SCID mice bearing MOLP-8 xenograft. Data points represent group mean body weight. Error bars represent standard error of the mean (SEM).

FIG. 4: Tumor volume trace after administering BT66BDC1 to female CB17-SCID mice bearing MOLP-8 xenograft. Data points represent group mean, error bars represent standard error of the mean (SEM).

### DETAILED DESCRIPTION OF THE INVENTION

In one embodiment, said loop sequences comprise 2, 3, 5, 6 or 7 amino acids.

In a further embodiment, said loop sequences comprise three cysteine residues separated by two loop sequences one of which consists of 2 amino acids and the other of which consists of 7 amino acids.

In a further embodiment, said loop sequences comprise three cysteine residues separated by two loop sequences one of which consists of 5 amino acids and the other of which consists of 6 amino acids.

In a further embodiment, said loop sequences comprise three cysteine residues separated by two loop sequences both of which consist of 3 amino acids.

In a further embodiment, said loop sequences comprise three cysteine residues separated by two loop sequences both of which consist of 5 amino acids.

In a further embodiment, said loop sequences comprise three cysteine residues separated by two loop sequences both of which consist of 6 amino acids.

In one embodiment, said peptide ligand comprises an amino acid sequence selected from:

(SEQ ID NO: 87)
Cᵢ-F-X₁-L-D-X₂-X₃-Cᵢᵢ-F-D-X₄-X₅-X₆-X₇-Cᵢᵢᵢ;
(SEQ ID NO: 88)
Cᵢ-I-R/N-Y-G/A-D/N-I-Cᵢᵢ-X₁-D/H-P/T-D/E-X₂-X₃-
Cᵢᵢᵢ;
(SEQ ID NO: 89)
Cᵢ-F-X₁-L-D-G-E-Cᵢᵢ-F-X₂-X₃-G/P-X₄-X₅-Cᵢᵢᵢ;
(SEQ ID NO: 2)
CᵢVNFGSVCᵢᵢWDPDSRCᵢᵢᵢ;
(SEQ ID NO: 90);
Cᵢ-X₁-X₂-Cᵢᵢ-A-D-F/M-P-I-X₃-X₄-Cᵢᵢᵢ;
(SEQ ID NO: 74)
CᵢDYCᵢᵢVRLGLTGCᵢᵢᵢ;
(SEQ ID NO: 75)
CᵢGWCᵢᵢSDQIDGFCᵢᵢᵢ;
(SEQ ID NO: 76)
CᵢAWCᵢᵢSDPIDGFCᵢᵢᵢ;
(SEQ ID NO: 77)
CᵢDWCᵢᵢIDPGVSFCᵢᵢᵢ;
(SEQ ID NO: 78)
CᵢSWCᵢᵢVDDGLPFCᵢᵢᵢ;
(SEQ ID NO: 79)
CᵢTWCᵢᵢVDDGLSFCᵢᵢᵢ;
(SEQ ID NO: 80)
CᵢTWCᵢᵢVDDETWNCᵢᵢᵢ;
(SEQ ID NO: 81)
CᵢDYCᵢᵢIRLGLTGCᵢᵢᵢ;
(SEQ ID NO: 82)
CᵢDWCᵢᵢTDNIPGICᵢᵢᵢ;
(SEQ ID NO: 91);
Cᵢ-A/N-W/F-L-Cᵢᵢ-P/D-N/D-L-Cᵢᵢᵢ;
(SEQ ID NO: 92)
Cᵢ-D-F-T-M-P-Cᵢᵢ-X₁-X₂-W-X₃-X₄-Cᵢᵢᵢ;
and
(SEQ ID NO: 28)
CᵢIFDYDCᵢᵢDAWSACᵢᵢᵢ;

wherein X₁-X₆ represent any amino acid residue, X₇ is either absent or represents any amino acid, and Cᵢ, Cᵢᵢ and Cᵢᵢᵢ represent first, second and third cysteine residues, respectively or a pharmaceutically acceptable salt thereof.

In one embodiment, said loop sequences either comprise three cysteine residues separated by two loop sequences both of which consist of 6 amino acids or one of which consists of 5 amino acids and the other of which consists of 6 amino acids, and said peptide ligand comprises an amino acid sequence selected from:

(SEQ ID NO: 87)
Cᵢ-F-X₁-L-D-X₂-X₃-Cᵢᵢ-F-D-X₄-X₅-X₆-X₇-Cᵢᵢᵢ;
(SEQ ID NO: 88)
Cᵢ-I-R/N-Y-G/A-D/N-I-Cᵢᵢ-X₁-D/H-P/T-D/E-X₂-X₃-
Cᵢᵢᵢ;
(SEQ ID NO: 89)
Cᵢ-F-X₁-L-D-G-E-Cᵢᵢ-F-X₂-X₃-G/P-X₄-X₅-Cᵢᵢᵢ;
and
(SEQ ID NO: 2)
CᵢVNFGSVCᵢᵢWDPDSRCᵢᵢᵢ;

wherein X₁-X₆ represent any amino acid residue, X₇ is either absent or represents any amino acid, and Cᵢ, Cᵢᵢ and Cᵢᵢᵢ represent first, second and third cysteine residues, respectively or a pharmaceutically acceptable salt thereof.

In one embodiment, said loop sequences comprise three cysteine residues separated by two loop sequences the first of which consists of 2 amino acids and the second of which consists of 7 amino acids, and said peptide ligand comprises an amino acid sequence selected from:

(SEQ ID NO: 90);
Cᵢ-X₁-X₂-Cᵢᵢ-A-D-F/M-P-I-X₃-X₄-Cᵢᵢᵢ;
(SEQ ID NO: 74)
CᵢDYCᵢᵢVRLGLTGCᵢᵢᵢ;
(SEQ ID NO: 75)
CᵢGWCᵢᵢSDQIDGFCᵢᵢᵢ;
(SEQ ID NO: 76)
CᵢAWCᵢᵢSDPIDGFCᵢᵢᵢ;
(SEQ ID NO: 77)
CᵢDWCᵢᵢIDPGVSFCᵢᵢᵢ;
(SEQ ID NO: 78)
CᵢSWCᵢᵢVDDGLPFCᵢᵢᵢ;
(SEQ ID NO: 79)
CᵢTWCᵢᵢVDDGLSFCᵢᵢᵢ;
(SEQ ID NO: 80)
CᵢTWCᵢᵢVDDETWNCᵢᵢᵢ;
SEQ ID NO: 81)
CᵢDYCᵢᵢIRLGLTGCᵢᵢᵢ;
and
(SEQ ID NO: 82)
CᵢDWCᵢᵢTDNIPGICᵢᵢᵢ;

wherein X₁-X₄ represent any amino acid residue, and Cᵢ, Cᵢᵢ and Cᵢᵢᵢ represent first, second and third cysteine residues, respectively or a pharmaceutically acceptable salt thereof.

In one embodiment, said loop sequences comprise three cysteine residues separated by two loop sequences both of which consist of 3 amino acids, and said peptide ligand comprises an amino acid sequence selected from:

(SEQ ID NO: 91)
Cᵢ-A/N-W/F-L-Cᵢᵢ-P/D-N/D-L-Cᵢᵢᵢ;

wherein Cᵢ, Cᵢᵢ and Cᵢᵢᵢ represent first, second and third cysteine residues, respectively or a pharmaceutically acceptable salt thereof.

In one embodiment, said loop sequences comprise three cysteine residues separated by two loop sequences both of which consist of 5 amino acids, and said peptide ligand comprises an amino acid sequence selected from:

(SEQ ID NO: 92)
Cᵢ-D-F-T-M-P-Cᵢᵢ-X₁-X₂-W-X₃-X₄-Cᵢᵢᵢ;
and
(SEQ ID NO: 28)
CᵢIFDYDCᵢᵢDAWSACᵢᵢᵢ;

wherein X₁-X₄ represent any amino acid residue, and Cᵢ, Cᵢᵢ and Cᵢᵢᵢ represent first, second and third cysteine residues, respectively or a pharmaceutically acceptable salt thereof.

In a further embodiment, the peptide ligand of Cᵢ—F—X₁-L-D-X₂-X₃—Cᵢᵢ—F-D-X₄—X₅—X₆—X₇—Cᵢᵢᵢ (SEQ ID NO: 87) comprises an amino acid sequence selected from:

(SEQ ID NO: 1)
CᵢFELDGTCᵢᵢFDWAQECᵢᵢᵢ;
(SEQ ID NO: 29)
CᵢFWLDGECᵢᵢFDWNHECᵢᵢᵢ;
(SEQ ID NO: 30)
CᵢFHLDGECᵢᵢFDLENTCᵢᵢᵢ;
(SEQ ID NO: 32)
CᵢFSLDGECᵢᵢFDLSGECᵢᵢᵢ;
(SEQ ID NO: 33)
CᵢFTLDGECᵢᵢFDWTHECᵢᵢᵢ;
(SEQ ID NO: 34)
CᵢFKLDGVCᵢᵢFDLFHECᵢᵢᵢ;
(SEQ ID NO: 35)
CᵢFMLDGECᵢᵢFDLNKECᵢᵢᵢ;
(SEQ ID NO: 36)
CᵢFKLDGECᵢᵢFDWTHECᵢᵢᵢ;
(SEQ ID NO: 37)
CᵢFTLDGECᵢᵢFDWDAECᵢᵢᵢ;
(SEQ ID NO: 38)
CᵢFELDGSCᵢᵢFDFDHECᵢᵢᵢ;
(SEQ ID NO: 39)
CᵢFTLDGECᵢᵢFDVNRECᵢᵢᵢ;
(SEQ ID NO: 40)
CᵢFWLDHECᵢᵢFDWTHECᵢᵢᵢ;
(SEQ ID NO: 41)
CᵢFQLDGECᵢᵢFDIYRECᵢᵢᵢ;
(SEQ ID NO: 42)
CᵢFELDGNCᵢᵢFDWTHECᵢᵢᵢ;
(SEQ ID NO: 43)
CᵢFHLDGECᵢᵢFDYEHECᵢᵢᵢ;
(SEQ ID NO: 44)
CᵢFSLDGECᵢᵢFDIASECᵢᵢᵢ;
(SEQ ID NO: 45)
CᵢFQLDGECᵢᵢFDTSHECᵢᵢᵢ;
(SEQ ID NO: 46)
CᵢFSLDGACᵢᵢFDWTHECᵢᵢᵢ;
(SEQ ID NO: 47)
CᵢFVLDGECᵢᵢFDYYEECᵢᵢᵢ;
(SEQ ID NO: 48)
CᵢFRLDDECᵢᵢFDWTHECᵢᵢᵢ;
(SEQ ID NO: 49)
CᵢFRLDGVCᵢᵢFDLDDECᵢᵢᵢ;
(SEQ ID NO: 50)
CᵢFRLDGECᵢᵢFDMGQECᵢᵢᵢ;
(SEQ ID NO: 51)
CᵢFTLDGACᵢᵢFDLDGECᵢᵢᵢ;
(SEQ ID NO: 52)
CᵢFTLDGQCᵢᵢFDWTHECᵢᵢᵢ;
(SEQ ID NO: 53)
CᵢFLLDGECᵢᵢFDWMQECᵢᵢᵢ;
(SEQ ID NO: 54)
CᵢFELDGDCᵢᵢFDWTHECᵢᵢᵢ;
(SEQ ID NO: 55)
CᵢFTLDGTCᵢᵢFDWTHECᵢᵢᵢ;
(SEQ ID NO: 56)
CᵢFHLDGVCᵢᵢFDWTHECᵢᵢᵢ;
(SEQ ID NO: 57)
CᵢFYLDGTCᵢᵢFDWTHECᵢᵢᵢ;
(SEQ ID NO: 58)
CᵢFLLDGECᵢᵢFDWAQECᵢᵢᵢ;
(SEQ ID NO: 59)
CᵢFHLDGECᵢᵢFDLAKTCᵢᵢᵢ;
(SEQ ID NO: 62)
CᵢFTLDGECᵢᵢFDLDGWCᵢᵢᵢ;
(SEQ ID NO: 63)
CᵢFLLDGECᵢᵢFDLIGECᵢᵢᵢ;
(SEQ ID NO: 67)
CᵢFWLDGECᵢᵢFDLGGQCᵢᵢᵢ;
(SEQ ID NO: 68)
CᵢFELDGECᵢᵢFDLDNQCᵢᵢᵢ;
(SEQ ID NO: 69)
CᵢFWLDGECᵢᵢFDLYGGCᵢᵢᵢ;
(SEQ ID NO: 70)
CᵢFRLDGECᵢᵢFDISNECᵢᵢᵢ;
(SEQ ID NO: 72)
CᵢFWLDGECᵢᵢFDFGGCᵢᵢᵢ;
and
(SEQ ID NO: 73)
CᵢFTLDGACᵢᵢFDWTHECᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ—F—X₁-L-D-X₂-X₃—Cᵢᵢ—F-D-X₄—X₅—X₆—X₇—Cᵢᵢᵢ (SEQ ID NO: 87) comprises an amino acid sequence selected from:
A-(SEQ ID NO: 1)-A (herein referred to as 66-01-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K (herein referred to as 66-08-01-N005);
Ac-(SEQ ID NO: 29)-A-Sar₆-K(DOTA) (herein referred to as 66-08-01-N016);
A-(SEQ ID NO: 30)-A (herein referred to as 66-08-44-N001);
Ac-A-(SEQ ID NO: 30)-A-Sar₆-K(Biot) (herein referred to as 66-08-01-N003);
A-(SEQ ID NO: 32)-A (herein referred to as 66-08-02-N001);
A-(SEQ ID NO: 33)-A (herein referred to as 66-08-03-N001);
A-(SEQ ID NO: 34)-A (herein referred to as 66-08-04-N001);
Ac-A-(SEQ ID NO: 35)-A (herein referred to as 66-08-05-N001);
A-(SEQ ID NO: 36)-A (herein referred to as 66-08-06-N001);
A-(SEQ ID NO: 37)-A (herein referred to as 66-08-07-N001);
A-(SEQ ID NO: 38)-A (herein referred to as 66-08-09-N001);
A-(SEQ ID NO: 39)-A (herein referred to as 66-08-13-N001);
A-(SEQ ID NO: 40)-A (herein referred to as 66-08-15-N001);
A-(SEQ ID NO: 41)-A (herein referred to as 66-08-17-N001);
A-(SEQ ID NO: 42)-A (herein referred to as 66-08-18-N001);
A-(SEQ ID NO: 43)-A (herein referred to as 66-08-20-N001);
A-(SEQ ID NO: 44)-A (herein referred to as 66-08-22-N001);
A-(SEQ ID NO: 45)-A (herein referred to as 66-08-24-N001);
A-(SEQ ID NO: 46)-A (herein referred to as 66-08-26-N001);
A-(SEQ ID NO: 47)-A (herein referred to as 66-08-27-N001);
A-(SEQ ID NO: 48)-A (herein referred to as 66-08-28-N001);
A-(SEQ ID NO: 49)-A (herein referred to as 66-08-29-N001);
A-(SEQ ID NO: 50)-A (herein referred to as 66-08-30-N001);
A-(SEQ ID NO: 51)-A (herein referred to as 66-08-31-N001);
A-(SEQ ID NO: 52)-A (herein referred to as 66-08-32-N001);
A-(SEQ ID NO: 53)-A (herein referred to as 66-08-33-N001);
A-(SEQ ID NO: 54)-A (herein referred to as 66-08-34-N001);
A-(SEQ ID NO: 55)-A (herein referred to as 66-08-35-N001);
A-(SEQ ID NO: 56)-A (herein referred to as 66-08-36-N001);
A-(SEQ ID NO: 57)-A (herein referred to as 66-08-41-N001);
A-(SEQ ID NO: 58)-A (herein referred to as 66-08-43-N001);
A-(SEQ ID NO: 59)-A (herein referred to as 66-08-45-N001);
A-(SEQ ID NO: 62)-A (herein referred to as 66-08-48-N001);
A-(SEQ ID NO: 63)-A (herein referred to as 66-08-49-N001);
A-(SEQ ID NO: 67)-A (herein referred to as 66-08-53-N001);
A-(SEQ ID NO: 68)-A (herein referred to as 66-08-54-N001);
A-(SEQ ID NO: 69)-A (herein referred to as 66-08-55-N001);
A-(SEQ ID NO: 70)-A (herein referred to as 66-08-56-N001);
A-(SEQ ID NO: 72)-A (herein referred to as 66-08-58-N001); and
A-(SEQ ID NO: 73)-A (herein referred to as 66-08-N002).

In a further embodiment, the peptide ligand of Cᵢ—I—R/N—Y-G/A-D/N—I—Cᵢᵢ—X₁-D/H—P/T-D/E-X₂—X₃—Cᵢᵢᵢ (SEQ ID NO: 88) comprises an amino acid sequence selected from:

(SEQ ID NO: 83)
CᵢIRYGDICᵢᵢYDPDHSCᵢᵢᵢ;
(SEQ ID NO: 84)
CᵢIRYGDICᵢᵢFHPDYTCᵢᵢᵢ;
and
(SEQ ID NO: 85)
CᵢINYANICᵢᵢLDTEKMCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ—I—R/N—Y-G/A-D/N—I—Cᵢᵢ—X₁-D/H—P/T-D/E-X₂—X₃—Cᵢᵢᵢ (SEQ ID NO: 88) comprises an amino acid sequence selected from:
A-(SEQ ID NO: 83)-A (herein referred to as 66-17-01-N001);
A-(SEQ ID NO: 84)-A (herein referred to as 66-17-N001); and
A-(SEQ ID NO: 85)-A (herein referred to as 66-18-N001).

In a further embodiment, the peptide ligand of Cᵢ—F—X₁-L-D-G-E-Cᵢᵢ—F—X₂-X₃-G/P—X₄—X₅—Cᵢᵢᵢ (SEQ ID NO: 89) comprises an amino acid sequence selected from:

(SEQ ID NO: 60)
CᵢFELDGECᵢᵢFHFGEPCᵢᵢᵢ;
(SEQ ID NO: 61)
CᵢFVLDGECᵢᵢFEIGERCᵢᵢᵢ;
(SEQ ID NO: 64)
CᵢFELDGECᵢᵢFSFPGTCᵢᵢᵢ;
(SEQ ID NO: 65)
CᵢFELDGECᵢᵢFSWPYPCᵢᵢᵢ;
(SEQ ID NO: 66)
CᵢFTLDGECᵢᵢFLLGENCᵢᵢᵢ;
and
(SEQ ID NO: 71)
CᵢFELDGECᵢᵢFNIGSKCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ—F—X₁-L-D-G-E-Cᵢᵢ—F—X₂-X₃-G/P—X₄—X₅—Cᵢᵢᵢ (SEQ ID NO: 89) comprises an amino acid sequence selected from:
A-(SEQ ID NO: 60)-A (herein referred to as 66-08-46-N001);
A-(SEQ ID NO: 61)-A (herein referred to as 66-08-47-N001);
A-(SEQ ID NO: 64)-A (herein referred to as 66-08-50-N001);
A-(SEQ ID NO: 65)-A (herein referred to as 66-08-51-N001);
A-(SEQ ID NO: 66)-A (herein referred to as 66-08-52-N001); and
A-(SEQ ID NO: 71)-A (herein referred to as 66-08-57-N001).

In one embodiment, the peptide ligand of CᵢVNFGSVCᵢᵢWDPDSRCᵢᵢᵢ (SEQ ID NO: 2) comprises an amino acid sequence selected from:
A-(SEQ ID NO: 2)-A (herein referred to as 66-02-N002).

In a further embodiment, the peptide ligand of Cᵢ—X₁—X₂—Cᵢᵢ-A-D-F/M-P—I—X₃—X₄—Cᵢᵢᵢ (SEQ ID NO: 90) comprises a peptide ligand of Cᵢ—X₁—X₂—Cᵢᵢ-A-D-F—P—I—X₃—X₄—Cᵢᵢᵢ (SEQ ID NO: 91).

In a further embodiment, the peptide ligand of Cᵢ—X₁—X₂-A-D-F/M-Cᵢᵢ—P—I—X₃—X₄—Cᵢᵢᵢ (SEQ ID NO: 90) comprises an amino acid sequence selected from:

(SEQ ID NO: 3)
CᵢVPCᵢᵢADFPIWYCᵢᵢᵢ;
(SEQ ID NO: 4)
CᵢVPCᵢᵢADFPIWWCᵢᵢᵢ;
(SEQ ID NO: 5)
CᵢTPCᵢᵢADFPIWSCᵢᵢᵢ;
(SEQ ID NO: 6)
CᵢTPCᵢᵢADFPIHTCᵢᵢᵢ;
(SEQ ID NO: 7)
CᵢVHCᵢᵢADFPIWGCᵢᵢᵢ;
(SEQ ID NO: 8)
CᵢVPCᵢᵢADFPIWGCᵢᵢᵢ;
(SEQ ID NO: 9)
CᵢVMCᵢᵢADFPIWGCᵢᵢᵢ;
(SEQ ID NO: 10)
CᵢTPCᵢᵢADFPIWYCᵢᵢᵢ;
(SEQ ID NO: 11)
CᵢTPCᵢᵢADFPILTCᵢᵢᵢ;
(SEQ ID NO: 12)
CᵢVACᵢᵢADFPIWGCᵢᵢᵢ;
(SEQ ID NO: 13)
CᵢTPCᵢᵢADFPIYGCᵢᵢᵢ;
(SEQ ID NO: 14)
CᵢTPCᵢᵢADFPILDCᵢᵢᵢ;
(SEQ ID NO: 15)
CᵢVKCᵢᵢADFPIWGCᵢᵢᵢ;
(SEQ ID NO: 16)
CᵢTPCᵢᵢADMPIWTCᵢᵢᵢ;
(SEQ ID NO: 17)
CᵢIPCᵢᵢADFPIWGCᵢᵢᵢ;
(SEQ ID NO: 18)
CᵢIPCᵢᵢADFPISVCᵢᵢᵢ;
(SEQ ID NO: 19)
CᵢVPCᵢᵢADFPISFCᵢᵢᵢ;
(SEQ ID NO: 20)
CᵢIPCᵢᵢADFPISFCᵢᵢᵢ;
(SEQ ID NO: 21)
CᵢVPCᵢᵢADFPISVCᵢᵢᵢ;
(SEQ ID NO: 22)
CᵢVPCᵢᵢADFPIFTCᵢᵢᵢ;
(SEQ ID NO: 23)
CᵢIPCᵢᵢADFPIFTCᵢᵢᵢ;
and
(SEQ ID NO: 24)
CᵢTPCᵢᵢADFPIWGCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ—X₁—X₂-A-D-F/M-Cᵢᵢ—P—I—X₃—X₄—Cᵢᵢᵢ (SEQ ID NO: 90) or the peptide ligands of SEQ ID NOS: 74-82 comprise an amino acid sequence selected from:
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 4)-A (herein referred to as 66-03-00-N005);
A-(SEQ ID NO: 5)-A (herein referred to as 66-03-01-N001);
A-(SEQ ID NO: 6)-A (herein referred to as 66-03-02-N001);
A-(SEQ ID NO: 7)-A (herein referred to as 66-03-03-N001);
A-(SEQ ID NO: 8)-A (herein referred to as 66-03-04-N001);
Ac-(SEQ ID NO: 8) (herein referred to as 66-03-04-N002);
A-(SEQ ID NO: 9)-A (herein referred to as 66-03-05-N001);
A-(SEQ ID NO: 10)-A (herein referred to as 66-03-06-N001);
Ac-(SEQ ID NO: 10) (herein referred to as 66-03-06-N002);
A-(SEQ ID NO: 11)-A (herein referred to as 66-03-07-N001);
A-(SEQ ID NO: 12)-A (herein referred to as 66-03-08-N001);
A-(SEQ ID NO: 13)-A (herein referred to as 66-03-09-N001);
A-(SEQ ID NO: 14)-A (herein referred to as 66-03-10-N001);
A-(SEQ ID NO: 15)-A (herein referred to as 66-03-11-N001);
Ac-(SEQ ID NO: 15) (herein referred to as 66-03-11-N002);
A-(SEQ ID NO: 16)-A (herein referred to as 66-03-15-N001);
A-(SEQ ID NO: 17)-A (herein referred to as 66-03-16-N001);
A-(SEQ ID NO: 18)-A (herein referred to as 66-03-24-N003);
A-(SEQ ID NO: 19)-A (herein referred to as 66-03-25-N002);
A-(SEQ ID NO: 20)-A (herein referred to as 66-03-26-N003);
A-(SEQ ID NO: 21)-A (herein referred to as 66-03-27-N003);
A-(SEQ ID NO: 22)-A (herein referred to as 66-03-28-N002);
A-(SEQ ID NO: 23)-A (herein referred to as 66-03-29-N003);
A-(SEQ ID NO: 24)-A (herein referred to as 66-03-N002);
A-(SEQ ID NO: 24)-A-Sar₆-K(Biot) (herein referred to as 66-03-N003);
A-(SEQ ID NO: 74)-A (herein referred to as 66-09-N001);
A-(SEQ ID NO: 75)-A-Sar₆-K(Biotin) (herein referred to as 66-10-01-N001);
A-(SEQ ID NO: 76)-A-Sar₆-K(Biotin) (herein referred to as 66-10-02-N001);
A-(SEQ ID NO: 77)-A-Sar₆-K(Biotin) (herein referred to as 66-10-03-N001);
A-(SEQ ID NO: 78)-A-Sar₆-K(Biotin) (herein referred to as 66-10-04-N001);
A-(SEQ ID NO: 79)-A (herein referred to as 66-10-N001);
A-(SEQ ID NO: 80)-A (herein referred to as 66-11-N001);
A-(SEQ ID NO: 81)-A (herein referred to as 66-12-N001); and
A-(SEQ ID NO: 82)-A (herein referred to as 66-13-N001).

In a further embodiment, the peptide ligand of Cᵢ-A/N—W/F-L-Cᵢᵢ—P/D-N/D-L-Cᵢᵢᵢ (SEQ ID NO: 91) comprises an amino acid sequence selected from:

(SEQ ID NO: 31)
CᵢAWLCᵢᵢPNLCᵢᵢᵢ;
and
(SEQ ID NO: 86)
CᵢNFLCᵢᵢDDLCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-A/N—W/F-L-Cᵢᵢ—P/D-N/D-L-Cᵢᵢᵢ (SEQ ID NO: 91) comprises an amino acid sequence selected from:
SSQHG-(SEQ ID NO: 31)-A-Sar₆-K (herein referred to as 66-08-01-N006); and
RHSNY-(SEQ ID NO: 86)-A-Sar₆-K(Biotin) (herein referred to as 66-20-00-T001-N001).

In a further embodiment, the peptide ligand of Cᵢ-D-F-T-M-P—Cᵢᵢ—X₁—X₂—W—X₃—X₄—Cᵢᵢᵢ (SEQ ID NO: 92) comprises an amino acid sequence selected from:

(SEQ ID NO: 25)
CᵢDFTMPCᵢᵢENWKYCᵢᵢᵢ;
(SEQ ID NO: 26)
CᵢDFTMPCᵢᵢPNWNACᵢᵢᵢ;
and
(SEQ ID NO: 27)
CᵢDFTMPCᵢᵢQMWEQCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-D-F-T-M-P—Cᵢᵢ—X₁—X₂—W—X₃—X₄—Cᵢᵢᵢ (SEQ ID NO: 92) or CᵢIFDYDCᵢᵢDAWSACᵢᵢᵢ (SEQ ID NO: 28) comprises an amino acid sequence selected from:
A-(SEQ ID NO: 25)-A (herein referred to as 66-05-07-N001);
A-(SEQ ID NO: 26)-A (herein referred to as 66-05-09-N001);
A-(SEQ ID NO: 27)-A (herein referred to as 66-05-N002); and
A-(SEQ ID NO: 28)-A (herein referred to as 66-06-N002).

In one embodiment, the molecular scaffold is selected from 1,3,5-tris(bromomethyl)benzene (TBMB) and the peptide ligand comprises an amino acid sequence selected from:
A-(SEQ ID NO: 1)-A (herein referred to as 66-01-N002);
A-(SEQ ID NO: 2)-A (herein referred to as 66-02-N002);
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 4)-A (herein referred to as 66-03-00-N005);
A-(SEQ ID NO: 5)-A (herein referred to as 66-03-01-N001);
A-(SEQ ID NO: 6)-A (herein referred to as 66-03-02-N001);
A-(SEQ ID NO: 7)-A (herein referred to as 66-03-03-N001);
A-(SEQ ID NO: 8)-A (herein referred to as 66-03-04-N001);
Ac-(SEQ ID NO: 8) (herein referred to as 66-03-04-N002);
A-(SEQ ID NO: 9)-A (herein referred to as 66-03-05-N001);
A-(SEQ ID NO: 10)-A (herein referred to as 66-03-06-N001);
Ac-(SEQ ID NO: 10) (herein referred to as 66-03-06-N002);
A-(SEQ ID NO: 11)-A (herein referred to as 66-03-07-N001);
A-(SEQ ID NO: 12)-A (herein referred to as 66-03-08-N001);
A-(SEQ ID NO: 13)-A (herein referred to as 66-03-09-N001);
A-(SEQ ID NO: 14)-A (herein referred to as 66-03-10-N001);
A-(SEQ ID NO: 15)-A (herein referred to as 66-03-11-N001);
Ac-(SEQ ID NO: 15) (herein referred to as 66-03-11-N002);
A-(SEQ ID NO: 16)-A (herein referred to as 66-03-15-N001);
A-(SEQ ID NO: 17)-A (herein referred to as 66-03-16-N001);
A-(SEQ ID NO: 18)-A (herein referred to as 66-03-24-N003);
A-(SEQ ID NO: 19)-A (herein referred to as 66-03-25-N002);
A-(SEQ ID NO: 20)-A (herein referred to as 66-03-26-N003);
A-(SEQ ID NO: 21)-A (herein referred to as 66-03-27-N003);
A-(SEQ ID NO: 22)-A (herein referred to as 66-03-28-N002);
A-(SEQ ID NO: 23)-A (herein referred to as 66-03-29-N003);
A-(SEQ ID NO: 24)-A (herein referred to as 66-03-N002);
A-(SEQ ID NO: 24)-A-Sar₆-K(Biot) (herein referred to as 66-03-N003);
A-(SEQ ID NO: 25)-A (herein referred to as 66-05-07-N001);
A-(SEQ ID NO: 26)-A (herein referred to as 66-05-09-N001);
A-(SEQ ID NO: 27)-A (herein referred to as 66-05-N002);
A-(SEQ ID NO: 28)-A (herein referred to as 66-06-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K (herein referred to as 66-08-01-N005);
Ac-(SEQ ID NO: 29)-A-Sar₆-K(DOTA) (herein referred to as 66-08-01-N016);
Ac-A-(SEQ ID NO: 30)-A-Sar₆-K(Biot) (herein referred to as 66-08-01-N003);
A-(SEQ ID NO: 30)-A (herein referred to as 66-08-44-N001);
SSQHG-(SEQ ID NO: 31)-A-Sar₆-K (herein referred to as 66-08-01-N006);
A-(SEQ ID NO: 32)-A (herein referred to as 66-08-02-N001);
A-(SEQ ID NO: 33)-A (herein referred to as 66-08-03-N001);
A-(SEQ ID NO: 34)-A (herein referred to as 66-08-04-N001);
Ac-A-(SEQ ID NO: 35)-A (herein referred to as 66-08-05-N001);
A-(SEQ ID NO: 36)-A (herein referred to as 66-08-06-N001);
A-(SEQ ID NO: 37)-A (herein referred to as 66-08-07-N001);
A-(SEQ ID NO: 38)-A (herein referred to as 66-08-09-N001);
A-(SEQ ID NO: 39)-A (herein referred to as 66-08-13-N001);
A-(SEQ ID NO: 40)-A (herein referred to as 66-08-15-N001);
A-(SEQ ID NO: 41)-A (herein referred to as 66-08-17-N001);
A-(SEQ ID NO: 42)-A (herein referred to as 66-08-18-N001);
A-(SEQ ID NO: 43)-A (herein referred to as 66-08-20-N001);
A-(SEQ ID NO: 44)-A (herein referred to as 66-08-22-N001);
A-(SEQ ID NO: 45)-A (herein referred to as 66-08-24-N001);
A-(SEQ ID NO: 46)-A (herein referred to as 66-08-26-N001);
A-(SEQ ID NO: 47)-A (herein referred to as 66-08-27-N001);
A-(SEQ ID NO: 48)-A (herein referred to as 66-08-28-N001);
A-(SEQ ID NO: 49)-A (herein referred to as 66-08-29-N001);
A-(SEQ ID NO: 50)-A (herein referred to as 66-08-30-N001);
A-(SEQ ID NO: 51)-A (herein referred to as 66-08-31-N001);
A-(SEQ ID NO: 52)-A (herein referred to as 66-08-32-N001);
A-(SEQ ID NO: 53)-A (herein referred to as 66-08-33-N001);
A-(SEQ ID NO: 54)-A (herein referred to as 66-08-34-N001);
A-(SEQ ID NO: 55)-A (herein referred to as 66-08-35-N001);
A-(SEQ ID NO: 56)-A (herein referred to as 66-08-36-N001);
A-(SEQ ID NO: 57)-A (herein referred to as 66-08-41-N001);
A-(SEQ ID NO: 58)-A (herein referred to as 66-08-43-N001);
A-(SEQ ID NO: 59)-A (herein referred to as 66-08-45-N001);
A-(SEQ ID NO: 60)-A (herein referred to as 66-08-46-N001);
A-(SEQ ID NO: 61)-A (herein referred to as 66-08-47-N001);
A-(SEQ ID NO: 62)-A (herein referred to as 66-08-48-N001);
A-(SEQ ID NO: 63)-A (herein referred to as 66-08-49-N001);
A-(SEQ ID NO: 64)-A (herein referred to as 66-08-50-N001);
A-(SEQ ID NO: 65)-A (herein referred to as 66-08-51-N001);
A-(SEQ ID NO: 66)-A (herein referred to as 66-08-52-N001);
A-(SEQ ID NO: 67)-A (herein referred to as 66-08-53-N001);
A-(SEQ ID NO: 68)-A (herein referred to as 66-08-54-N001);
A-(SEQ ID NO: 69)-A (herein referred to as 66-08-55-N001);
A-(SEQ ID NO: 70)-A (herein referred to as 66-08-56-N001);
A-(SEQ ID NO: 71)-A (herein referred to as 66-08-57-N001);
A-(SEQ ID NO: 72)-A (herein referred to as 66-08-58-N001);
A-(SEQ ID NO: 73)-A (herein referred to as 66-08-N002);
A-(SEQ ID NO: 74)-A (herein referred to as 66-09-N001);
A-(SEQ ID NO: 75)-A-Sar₆-K(Biotin) (herein referred to as 66-10-01-N001);
A-(SEQ ID NO: 76)-A-Sar₆-K(Biotin) (herein referred to as 66-10-02-N001);
A-(SEQ ID NO: 77)-A-Sar₆-K(Biotin) (herein referred to as 66-10-03-N001);
A-(SEQ ID NO: 78)-A-Sar₆-K(Biotin) (herein referred to as 66-10-04-N001);
A-(SEQ ID NO: 79)-A (herein referred to as 66-10-N001);
A-(SEQ ID NO: 80)-A (herein referred to as 66-11-N001);
A-(SEQ ID NO: 81)-A (herein referred to as 66-12-N001);
A-(SEQ ID NO: 82)-A (herein referred to as 66-13-N001);
A-(SEQ ID NO: 83)-A (herein referred to as 66-17-01-N001);
A-(SEQ ID NO: 84)-A (herein referred to as 66-17-N001);
A-(SEQ ID NO: 85)-A (herein referred to as 66-18-N001); and
RHSNY-(SEQ ID NO: 86)-A-Sar₆-K(Biotin) (herein referred to as 66-20-00-T001-N001).

In a further embodiment, the molecular scaffold is selected from 1,3,5-tris(bromomethyl)benzene (TBMB) and the peptide ligand comprises an amino acid sequence selected from:
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 7)-A (herein referred to as 66-03-03-N001);
A-(SEQ ID NO: 8)-A (herein referred to as 66-03-04-N001);
A-(SEQ ID NO: 10)-A (herein referred to as 66-03-06-N001);
A-(SEQ ID NO: 15)-A (herein referred to as 66-03-11-N001);
A-(SEQ ID NO: 22)-A (herein referred to as 66-03-28-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K(DOTA) (herein referred to as 66-08-01-N016); and
Ac-A-(SEQ ID NO: 30)-A-Sar₆-K(Biot) (herein referred to as 66-08-01-N003).

The scaffold/peptide ligands of this embodiment demonstrated superior CD38 competition binding as shown herein in Table 1.

In a yet further embodiment, the molecular scaffold is selected from 1,3,5-tris(bromomethyl)benzene (TBMB) and the peptide ligand comprises an amino acid sequence selected from:
(β-Ala)-Sar₁₀-A-(SEQ ID NO: 3) (herein referred to as 66-03-00-N006).

The scaffold/peptide ligand of this embodiment demonstrated superior integrin CD38 competition binding alone (as shown herein in Table 1) and when conjugated to the toxin DM-1 (as shown herein in Table 2).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

Nomenclature

Numbering

When referring to amino acid residue positions within the peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:

-Cᵢ-F₁-E₂-L₃-D₄-G₅-T₆-Cᵢᵢ-F₇-D₈-W₉-A₁₀-Q₁₁-E₁₂-
Cᵢᵢᵢ-(SEQ ID NO: 1).

For the purpose of this description, all bicyclic peptides are assumed to be cyclised with TBMB (1,3,5-tris(bromomethyl)benzene) and yielding a tri-substituted structure. Cyclisation with TBMB occurs on Cᵢ, Cᵢᵢ, and Cᵢᵢᵢ.

Molecular Format

N- or C-terminal extensions to the bicycle core sequence are added to the left or right side of the sequence, separated by a hyphen. For example, an N-terminal βAla-Sar10-Ala tail would be denoted as:

βAla-Sar10-A-(SEQ ID NO: X).

Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity. Certain peptide ligands of the invention demonstrate good selectivity over other CDs.

Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002.

Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Ca-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the CD38 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

Aromatic Molecular Scaffold

References herein to the term “aromatic molecular scaffold” refer to any molecular scaffold as defined herein which contains an aromatic carbocyclic or heterocyclic ring system.

It will be appreciated that the aromatic molecular scaffold may comprise an aromatic moiety. Examples of suitable aromatic moieties within the aromatic scaffold include biphenylene, terphenylene, naphthalene or anthracene.

It will also be appreciated that the aromatic molecular scaffold may comprise a heteroaromatic moiety. Examples of suitable heteroaromatic moieties within the aromatic scaffold include pyridine, pyrimidine, pyrrole, furan and thiophene.

It will also be appreciated that the aromatic molecular scaffold may comprise a halomethylarene moiety, such as a bis(bromomethyl)benzene, a tris(bromomethyl)benzene, a tetra(bromomethyl)benzene or derivatives thereof.

Non-limiting examples of aromatic molecular scaffolds include: bis-, tris-, or tetra(halomethyl)benzene; bis-, tris-, or tetra(halomethyl)pyridine; bis-, tris-, or tetra(halomethyl)pyridazine; bis-, tris-, or tetra(halomethyl)pyrimidine; bis-, tris-, or tetra(halomethyl)pyrazine; bis-, tris-, or tetra(halomethyl)-1,2,3-triazine; bis-, tris-, or tetra-halomethyl)-1,2,4-triazine; bis-, tris-, or tetra(halomethyl)pyrrole, -furan, -thiophene; bis-, tris-, or tetra(halomethyl)imidazole, -oxazole, -thiazol; bis-, tris-, or tetra(halomethyl)-3H-pyrazole, -isooxazole, -isothiazol; bis-, tris-, or tetra(halomethyl)biphenylene; bis-, tris-, or tetra(halomethyl)terphenylene; 1,8-bis(halomethyl)naphthalene; bis-, tris-, or tetra(halomethyl)anthracene; and bis-, tris-, or tetra(2-halomethylphenyl)methane.

More specific examples of aromatic molecular scaffolds include: 1,2-bis(halomethyl)benzene; 3,4-bis(halomethyl)pyridine; 3,4-bis(halomethyl)pyridazine; 4,5-bis(halomethyl)pyrimidine; 4,5-bis(halomethyl)pyrazine; 4,5-bis(halomethyl)-1,2,3-triazine; 5,6-bis(halomethyl)-1,2,4-triazine; 3,4-bis(halomethyl)pyrrole, -furan, -thiophene and other regioisomers; 4,5-bis(halomethyl)imidazole, -oxazole, -thiazol; 4,5-bis(halomethyl)-3H-pyrazole, -isooxazole, -isothiazol; 2,2′-bis(halomethyl)biphenylene; 2,2″-bis(halomethyl)terphenylene; 1,8-bis(halomethyl)naphthalene; 1,10-bis(halomethyl)anthracene; bis(2-halomethylphenyl)methane; 1,2,3-tris(halomethyl)benzene; 2,3,4-tris(halomethyl)pyridine; 2,3,4-tris(halomethyl)pyridazine; 3,4,5-tris(halomethyl)pyrimidine; 4,5,6-tris(halomethyl)-1,2,3-triazine; 2,3,4-tris(halomethyl)pyrrole, -furan, -thiophene; 2,4,5-bis(halomethyl)imidazole, -oxazole, -thiazol; 3,4,5-bis(halomethyl)-1H-pyrazole, -isooxazole, -isothiazol; 2,4,2′-tris(halomethyl)biphenylene; 2,3′,2″-tris(halomethyl)terphenylene; 1,3,8-tris(halomethyl)naphthalene; 1,3,10-tris(halomethyl)anthracene; bis(2-halomethylphenyl)methane; 1,2,4,5-tetra(halomethyl)benzene; 1,2,4,5-tetra(halomethyl)pyridine; 2,4,5,6-tetra(halomethyl)pyrimidine; 2,3,4,5-tetra(halomethyl)pyrrole, -furan, -thiophene; 2,2′,6,6′-tetra(halomethyl)biphenylene; 2,2″,6,6″-tetra(halomethyl) terphenylene; 2,3,5,6-tetra(halomethyl)naphthalene and 2,3,7,8-tetra(halomethyl)anthracene; and bis(2,4-bis(halomethyl)phenyl)methane.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of tris(bromomethyl)benzene, especially 1,3,5-tris(bromomethyl)benzene (‘TBMB’), or a derivative thereof.

In one embodiment, the molecular scaffold is 2,4,6-tris(bromomethyl)mesitylene. This molecule is similar to 1,3,5-tris(bromomethyl)benzene but contains three additional methyl groups attached to the benzene ring. This has the advantage that the additional methyl groups may form further contacts with the polypeptide and hence add additional structural constraint.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene (the scaffold reactive group exemplified by TBMB) or iodoacetamide. Other scaffold reactive groups that are used to selectively couple compounds to cysteines in proteins are maleimides, αβ unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an α-halomethylcarbonyl containing compound is N,N′,N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and 35 Harrison, Biochemical Society Transactions (2007) Volume 35, part 4, p821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p10444), the 18 amino acid ‘model amphipathic peptide’ (Oehlke et al (1998) Biochim Biophys Acts Volume 1414 p127) and arginine rich regions of the HIV TAT protein. Non peptidic approaches include the use of small molecule mimics or SMOCs that can be easily attached to biomolecules (Okuyama et al (2007) Nature Methods Volume 4 p153). Other chemical strategies to add guanidinium groups to molecules also enhance cell penetration (Elson-Scwab et al (2007) J Biol Chem Volume 282 p13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

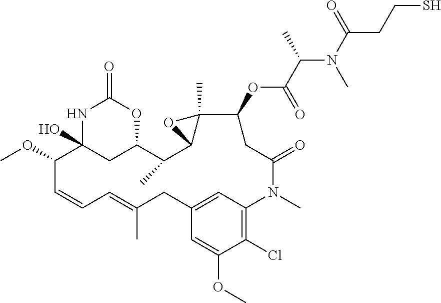 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@@H](OC)/C=C\C=C(/C)C2]

Monomethyl auristatin E (MMAE) is a synthetic antineoplastic agent and has the following structure:

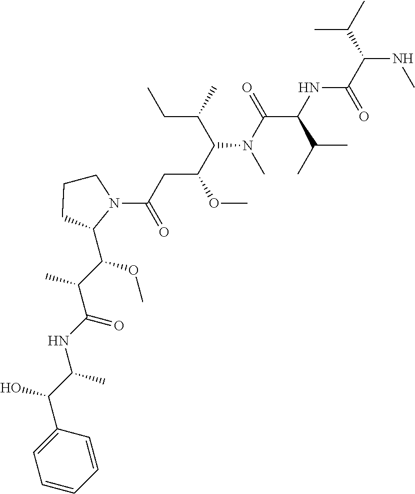

In one yet further particular embodiment of the invention, the cytotoxic agent is selected from maytansinoids (such as DM1). Data is presented herein in Table 2 which demonstrates the effects of peptide ligands conjugated to toxins containing DM1.

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

In one embodiment, the cytotoxic agent is DM1, the bicyclic peptide is (β-Ala)-Sar₁₀-A-(SEQ ID NO: 3) (herein referred to as 66-03-00-N006) and the conjugate comprises a compound of formula (I):

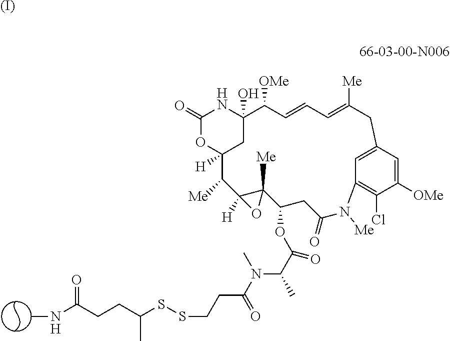 [SMILES: "(I)" ; CNC(=O)CCC(C)SSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

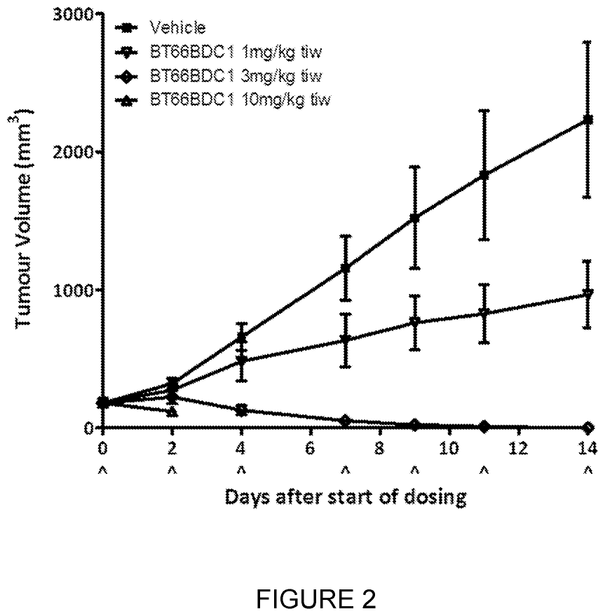

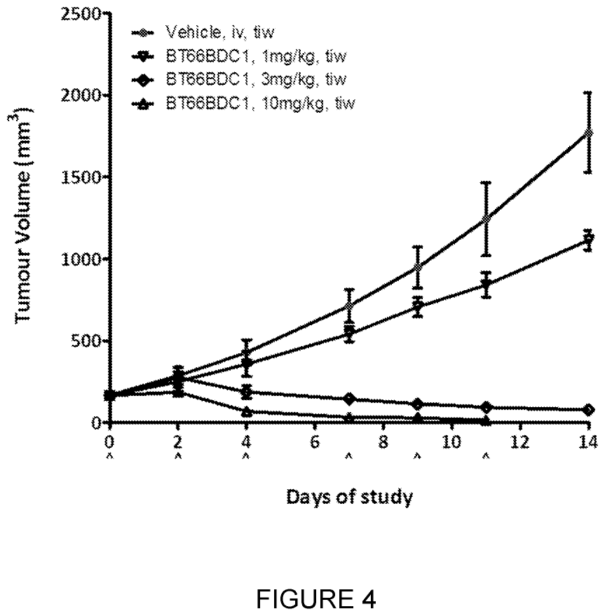

The BDC of formula (I) is known herein as BT66BDC-1. Data is presented herein which demonstrates excellent competition binding for BT66BDC-1 in the CD38 competition binding assay as shown in Table 2. In vivo data is also presented herein which demonstrates that 1 and 3 mg/kg of BT66BDC-1 produced dose-dependent antitumor activity in the MOLP-8 xenograft model as shown in Tables 9, 10 and FIG. 4. In vivo data is also presented herein which demonstrates that 3 mg/kg of BT66BDC-1 completely eradicated tumors by 14 days in the HT1080 xenograft model as shown in Tables 5, 6 and FIG. 2.

Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TBMB) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counter indications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Therapeutic Uses

The bicyclic peptides of the invention have specific utility as CD38 binding agents.

CD38 is a 45 kD type II transmembrane glycoprotein with a long C-terminal extracellular domain and a short N-terminal cytoplasmic domain. The CD38 protein is a bifunctional ectoenzyme that can catalyze the conversion of NAD+ into cyclic ADP-ribose (cADPR) and also hydrolyze cADPR into ADP-ribose. During ontogeny, CD38 appears on CD34+ committed stem cells and lineage-committed progenitors of lymphoid, erythroid and myeloid cells. CD38 expression persists mostly in the lymphoid lineage with varying expression levels at different stages of T and B cell development.

CD38 is upregulated in many hematopoeitic malignancies and in cell lines derived from various hematopoietic malignancies, including non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CML). On the other hand, most primitive pluripotent stem cells of the hematopoietic system are CD38-. CD38 expression in hematopoietic malignancies and its correlation with disease progression makes CD38 an attractive target for antibody therapy.

CD38 has been reported to be involved in Ca²⁺ mobilization (Morra et al. (1998) FASEB J. 12; 581-592; Zilber et al. (2000) Proc Natl Acad Sci USA 97, 2840-2845) and in the signal transduction through tyrosine phosphorylation of numerous signaling molecules, including phospholipase C-γ, ZAP-70, syk, and c-cbl, in lymphoid and myeloid cells or cell lines (Funaro et al. (1993) Eur J Immunol 23, 2407-2411; Morra et al. (1998), supra; Funaro et al. (1990) J Immunol 145, 2390-2396; Zubiaur et al. (1997) J Immunol 159, 193-205; Deaglio et al. (2003) Blood 102, 2146-2155; Todisco et al. (2000) Blood 95, 535-542; Konopleva et al. (1998) J Immunol 161, 4702-4708; Zilber et al. (2000) Proc Natl Acad Sci USA 97, 2840-2845; Kitanaka et al. (1997) J Immunol 159, 184-192; Kitanaka et al. (1999) J Immunol 162, 1952-1958; Mallone et al. (2001) Int Immunol 13, 397-409). On the basis of these observations, CD38 was proposed to be an important signaling molecule in the maturation and activation of lymphoid and myeloid cells during their normal development.

The exact role of CD38 in signal transduction and hematopoiesis is still not clear, especially since most of these signal transduction studies have used cell lines ectopically overexpressing CD38 and anti-CD38 monoclonal antibodies, which are non-physiological ligands. Because the CD38 protein has an enzymatic activity that produces cADPR, a molecule that can induce Ca²⁺ mobilization (Lee et al. (1989) J Biol Chem 264, 1608-1615; Lee and Aarhus (1991) Cell Regul 2, 203-209), it has been proposed that CD38 ligation by monoclonal antibodies triggers Ca²⁺ mobilization and signal transduction in lymphocytes by increasing production of cADPR (Lee et al. (1997) Adv Exp Med Biol 419, 411-419). Contrary to this hypothesis, the truncation and point-mutation analysis of CD38 protein showed that neither its cytoplasmic tail nor its enzymatic activity is necessary for the signaling mediated by anti-CD38 antibodies (Kitanaka et al. (1999) J Immunol 162, 1952-1958; Lund et al. (1999) J Immunol 162, 2693-2702; Hoshino et al. (1997) J Immunol 158, 741-747).

The best evidence for the function of CD38 comes from CD38−/− knockout mice, which have a defect in their innate immunity and a reduced T-cell dependent humoral response due to a defect in dendritic cell migration (Partida-Sanchez et al. (2004) Immunity 20, 279-291; Partida-Sanchez et al. (2001) Nat Med 7, 1209-1216). Nevertheless, it is not clear if the CD38 function in mice is identical to that in humans since the CD38 expression pattern during hematopoiesis differs greatly between human and mouse: a) unlike immature progenitor stem cells in humans, similar progenitor stem cells in mice express a high level of CD38 (Randall et al. (1996) Blood 87, 4057-4067; Dagher et al. (1998) Biol Blood Marrow Transplant 4, 69-74), b) while during the human B cell development, high levels of CD38 expression are found in germinal center B cells and plasma cells (Uckun (1990) Blood 76, 1908-1923; Kumagai et al. (1995) J Exp Med 181, 1101-1110), in the mouse, the CD38 expression levels in the corresponding cells are low (Oliver et al. (1997) J Immunol 158, 1108-1115; Ridderstad and Tarlinton (1998) J Immunol 160, 4688-4695).

Several anti-human CD38 antibodies with different proliferative properties on various tumor cells and cell lines have been described in the literature. For example, a chimeric OKT10 antibody with mouse Fab and human IgG1 Fc mediates antibody-dependent cell-mediated cytotoxicity (ADCC) very efficiently against lymphoma cells in the presence of peripheral blood mononuclear effector cells from either MM patients or normal individuals (Stevenson et al. (1991) Blood 77, 1071-1079). A CDR-grafted humanized version of the anti-CD38 antibody AT13/5 has been shown to have potent ADCC activity against CD38-positive cell lines (U.S. patent application Ser. No. 09/797,941). Human monoclonal anti-CD38 antibodies have been shown to mediate the in vitro killing of CD38-positive cell lines by ADCC and/or complement-dependent cytotoxicity (CDC), and to delay the tumor growth in SCID mice bearing MM cell line RPMI-8226 (WO 2005/103083). On the other hand, several anti-CD38 antibodies, IB4, SUN-4B7, and OKT10, but not IB6, AT1, orAT2, induced the proliferation of peripheral blood mononuclear cells (PBMC) from normal individuals (Ausiello et al. (2000) Tissue Antigens 56, 539-547).

Some of the antibodies of the prior art have been shown to be able to trigger apoptosis in CD38+ B cells. However, they can only do so in the presence of stroma cells or stroma-derived cytokines. An agonistic anti-CD38 antibody (IB4) has been reported to prevent apoptosis of human germinal center (GC) B cells (Zupo et al. (1994) Eur J Immunol 24, 1218-1222), and to induce proliferation of KG-1 and HL-60 AML cells (Konopleva et al. (1998) J Immunol 161, 4702-4708), but induces apoptosis in Jurkat T lymphoblastic cells (Morra et al. (1998) FASEB J 12, 581-592). Another anti-CD38 antibody T16 induced apoptosis of immature lymphoid cells and leukemic lymphoblast cells from an ALL patient (Kumagai et al. (1995) J Exp Med 181, 1101-1110), and of leukemic myeloblast cells from AML patients (Todisco et al. (2000) Blood 95, 535-542), but T16 induced apoptosis only in the presence of stroma cells or stroma-derived cytokines (IL-7, IL-3, stem cell factor).

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by CD38.

According to a further aspect of the invention, there is provided a method of preventing, suppressing or treating a disease or disorder mediated by CD38, which comprises administering to a patient in need thereof an effector group and drug conjugate of the peptide ligand as defined herein.

In one embodiment, the CD38 is mammalian CD38. In a further embodiment, the mammalian CD38 is human CD38 (hCD38).

In one embodiment, the disease or disorder mediated by CD38 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CML).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

Materials and Methods

Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-tris(bromomethyl)benzene (TBMB, Sigma). For this, linear peptide was diluted with H₂O up to ˜35 mL, ˜500 μL of 100 mM TBMB in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Following lyophilisation, the modified peptide was purified as above, while replacing the Luna C8 with a Gemini C18 column (Phenomenex), and changing the acid to 0.1% trifluoroacetic acid. Pure fractions containing the correct TMB-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

Preparation of Bicyclic Peptide Drug Conjugate BT66BDC-1

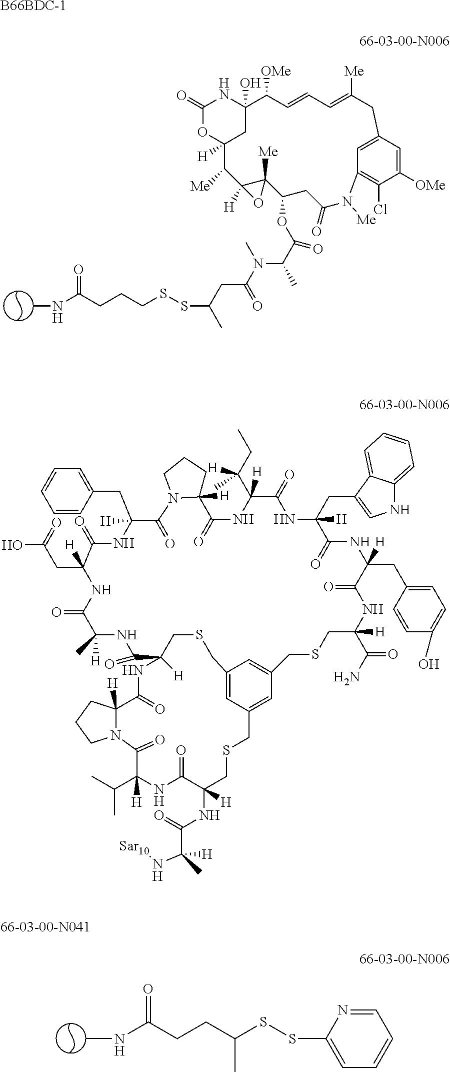

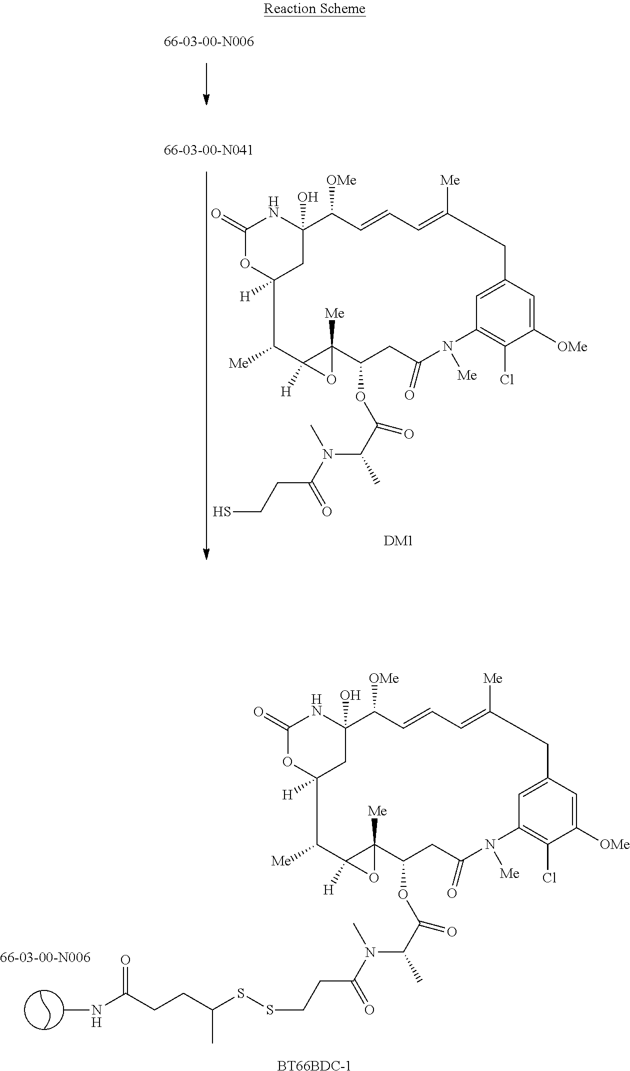

66-03-00-NO41 (56 mg) was dissolved in DMF (0.8 ml). A solution of DM1 (19 mg) in DMF (0.78 ml) was added followed by diisopropylethylamine (26 uL) and the mixture stirred at room temperature for 2 hour. Water (17.5 ml) was added and the mixture filtered before being loaded onto a Luna C18(3) prep HPLC column (250 mm×20 mm). The product was obtained by eluting with a gradient of acetonitrile (with 1% trfluoroacetic acid) and water (with 1% trfluoroacetic acid) 16%-55% over 50 minutes. Lyophilisation of the pure fractions gave 37.5 mg product LC/MS (ES⁺) calc for MH+ 3234.4; found 3234.4.

Biological Data

1. CD38 Competition Binding Assay

Affinity of the peptides of the invention for human CD38 (Ki) was determined using a fluorescence polarisation assay, using the method reported by Lea et al (Expert Opin Drug Discov. 2011 6(1): 17-3) and using the following fluorescently labelled peptides ACTPCADFPIWGCA-Sar₆-K(Fl) ((SEQ ID NO: 93)-Sar₆-K(Fl)) for TBMB derivatives where Fl is a fluorescein molecule.

The peptide ligands of the invention were tested in the above mentioned CD38 competition binding assay and the results are shown in Table 1:

TABLE 1
Biological Assay Data for Peptide Ligands of the Invention

| | Molecular | |
|---|---|---|
| Peptide | Scaffold | Ki (nM) |
| 66-01-N002 | TBMB | 1183.67 ± 286.2 |
| 66-02-N002 | TBMB | 3660 ± 319.09 |
| 66-03-00-N004 | TBMB | 35.33 ± 6.55 |
| 66-03-00-N005 | TBMB | 109.67 ± 51.03 |
| 66-03-00-N006 | TBMB | 43.67 ± 14.73 |
| 66-03-00-N007 | TBMB | 52.5 ± 26.46 |
| 66-03-00-N008 | TBMB | 73 n = 1 |
| 66-03-00-N009 | TBMB | 60.5 ± 10.78 |
| 66-03-01-N001 | TBMB | 471 n = 1 |
| 66-03-02-N001 | TBMB | 995 n = 1 |
| 66-03-03-N001 | TBMB | 61 ± 1.96 |
| 66-03-04-N001 | TBMB | 58.45 ± 33.88 |
| 66-03-04-N002 | TBMB | 152 n = 1 |
| 66-03-05-N001 | TBMB | 155 n = 1 |
| 66-03-06-N001 | TBMB | 64 ± 8.21 |
| 66-03-06-N002 | TBMB | 106 n = 1 |
| 66-03-07-N001 | TBMB | 257 n = 1 |
| 66-03-08-N001 | TBMB | 174 n = 1 |
| 66-03-09-N001 | TBMB | 1053 n = 1 |
| 66-03-10-N001 | TBMB | 1699 n = 1 |
| 66-03-11-N001 | TBMB | 41.3 ± 14.28 |
| 66-03-11-N002 | TBMB | 298 n = 1 |
| 66-03-15-N001 | TBMB | 274 n = 1 |
| 66-03-16-N001 | TBMB | 114 n = 1 |
| 66-03-24-N003 | TBMB | 170.5 ± 20.58 |
| 66-03-25-N002 | TBMB | 207.5 ± 26.46 |
| 66-03-26-N003 | TBMB | 160 ± 13.72 |
| 66-03-27-N003 | TBMB | 143 ± 27.44 |
| 66-03-28-N002 | TBMB | 44.5 ± 14.7 |
| 66-03-29-N003 | TBMB | 139 ± 25.48 |
| 66-03-N002 | TBMB | 198.33 ± 29.13 |
| 66-03-N003 | TBMB | 833 ± 356.71 |
| 66-05-07-N001 | TBMB | 2533 n = 1 |
| 66-05-09-N001 | TBMB | 3946 n = 1 |
| 66-05-N002 | TBMB | 4061.5 ± 75.46 |
| 66-06-N002 | TBMB | 7250 n = 1 |
| 66-08-01-N001 | TBMB | 43.3 ± 7.69 |
| 66-08-01-N003 | TBMB | 38.5 ± 10.78 |
| 66-08-01-N004 | TBMB | 99 n = 1 |
| 66-08-01-N005 | TBMB | 211 n = 1 |
| 66-08-01-N006 | TBMB | 106 n = 1 |
| 66-08-01-N016 | TBMB | 51 n = 1 |
| 66-08-02-N001 | TBMB | 305 n = 1 |
| 66-08-03-N001 | TBMB | 139 n = 1 |
| 66-08-04-N001 | TBMB | 3818 n = 1 |
| 66-08-05-N001 | TBMB | 1372 n = 1 |
| 66-08-06-N001 | TBMB | 677 n = 1 |
| 66-08-07-N001 | TBMB | 230 n = 1 |
| 66-08-09-N001 | TBMB | 611 n = 1 |
| 66-08-13-N001 | TBMB | 905 n = 1 |
| 66-08-15-N001 | TBMB | 258 n = 1 |
| 66-08-17-N001 | TBMB | 245 n = 1 |
| 66-08-18-N001 | TBMB | 980 n = 1 |
| 66-08-20-N001 | TBMB | 2164 n = 1 |
| 66-08-22-N001 | TBMB | 1045 n = 1 |
| 66-08-24-N001 | TBMB | 2970 n = 1 |
| 66-08-26-N001 | TBMB | 987 n = 1 |
| 66-08-27-N001 | TBMB | 1220 n = 1 |
| 66-08-28-N001 | TBMB | 1165 n = 1 |
| 66-08-29-N001 | TBMB | 988 n = 1 |
| 66-08-30-N001 | TBMB | 6949 n = 1 |
| 66-08-31-N001 | TBMB | 821 n = 1 |
| 66-08-32-N001 | TBMB | 794 n = 1 |
| 66-08-33-N001 | TBMB | 409 n = 1 |
| 66-08-34-N001 | TBMB | 1623 n = 1 |
| 66-08-35-N001 | TBMB | 744 n = 1 |
| 66-08-36-N001 | TBMB | 558 n = 1 |
| 66-08-41-N001 | TBMB | 1200 n = 1 |
| 66-08-43-N001 | TBMB | 131 n = 1 |
| 66-08-44-N001 | TBMB | 620 n = 1 |
| 66-08-45-N001 | TBMB | 8187 n = 1 |
| 66-08-46-N001 | TBMB | 1039.5 ± 169.54 |
| 66-08-47-N001 | TBMB | 237.5 ± 0.98 |
| 66-08-48-N001 | TBMB | 178 ± 60.76 |
| 66-08-49-N001 | TBMB | 1365 ± 150.92 |
| 66-08-50-N001 | TBMB | 196.5 ± 98.98 |
| 66-08-51-N001 | TBMB | 4750 ± 489.99 |
| 66-08-52-N001 | TBMB | 562.5 ± 210.7 |
| 66-08-53-N001 | TBMB | 119 ± 27.44 |
| 66-08-54-N001 | TBMB | 477 ± 121.52 |
| 66-08-55-N001 | TBMB | 105 ± 1.96 |
| 66-08-56-N001 | TBMB | 1149 ± 148.96 |
| 66-08-57-N001 | TBMB | 1101.5 ± 44.1 |
| 66-08-58-N001 | TBMB | 154 ± 17.64 |
| 66-08-N002 | TBMB | 1096 n = 1 |
| 66-09-N001 | TBMB | 1255 n = 1 |
| 66-10-01-N001 | TBMB | 2962 n = 1 |
| 66-10-02-N001 | TBMB | 2553 n = 1 |
| 66-10-03-N001 | TBMB | 3136 n = 1 |
| 66-10-04-N001 | TBMB | 3918 n = 1 |
| 66-10-N001 | TBMB | 270 n = 1 |
| 66-11-N001 | TBMB | 767 n = 1 |
| 66-12-N001 | TBMB | 520 n = 1 |
| 66-13-N001 | TBMB | 357 n = 1 |
| 66-17-01-N001 | TBMB | 260 n = 1 |
| 66-17-N001 | TBMB | 1166 n = 1 |
| 66-18-N001 | TBMB | 6894 n = 1 |
| 66-20-00-T001-N001 | TBMB | 2917 n = 1 |

The bicycle drug conjugate of BT66BDC-1 was tested in the above mentioned 0038 competition binding assay and the results are shown in Table 2:

TABLE 2
Biological Assay Data for Bicycle Drug Conjugates of the Invention

| Bicycle Drug | | |
|---|---|---|
| Conjugates | Structure | Ki (nM) |
| BT66BDC-1 | 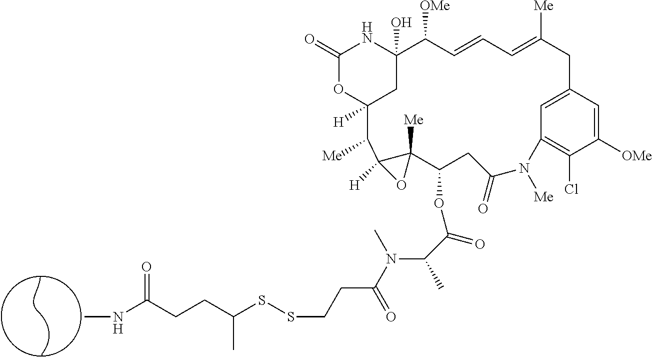 [SMILES: CNC(=O)CCC(C)SSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C] | 144 |
| | 66-03-00-N006 | |

*n = 1

2. In Viva Efficacy Test of BT66BDC1 in Treatment of HT1080 Xenograft in BALB/c Nude Mice

2.1 Study Objective

The objective of the research was to evaluate the in vivo anti-tumor efficacy of BT66BDC1 in treatment of HT1080 xenograft in BALE/c nude mice.

2.2 Experimental Design

TABLE 3

| | | | | Dosing | | |
|---|---|---|---|---|---|---|
| | | | Dosage | Volume | Dosing | |
| Gr | n | Treatment | (mg/kg) | (ml/g) | Route | Schedule |
| 1 | 3 | Vehicle | — | 10 | i.v. | tiw*2 weeks |
| 2 | 3 | BT66BDC1 | 1 | 10 | i.v. | tiw*2 weeks |
| 3 | 3 | BT66BDC1 | 3 | 10 | i.v. | tiw*2 weeks |
| 4 | 3 | BT66BDC1 | 10 | 10 | i.v. | tiw*2 weeks |

2.3 Materials

2.3.1 Animals and Housing Condition

2.3.1.1 Animals
Species: Mus Musculus
Strain: Balb/c nude
Age: 6-8 weeks
Sex: female
Body weight: 18-22 g
Number of animals: 12 mice plus spare
Animal supplier: Shanghai LC Laboratory Animal Co., LTD.

2.3.1.2 Housing Conditions
The mice were kept in individual ventilation cages at constant temperature and humidity with 3 animals in each cage.
   Temperature: 20˜26° C.
   Humidity 40-70%.
Cages: Made of polycarbonate. Size: 300 mm×180 mm×150 mm. The bedding material was corn cob, which was changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, the date received, treatment, study number, group number and the starting date of the treatment.
Animal identification: Animals were marked by ear coding.

2.3.2 Test and Positive Control Articles
Product identification: BT66BDC1
Physical description: Lyophilised powder
Molecular weight: 3234.4
Package and storage condition: stored at −80° C.

2.4 Experimental Methods and Procedures

2.4.1 Cell Culture

The HT1080 tumor cells were maintained in vitro as a monolayer culture in EMEM medium supplemented with 10% heat inactivated fetal bovine serum at 37° C. in an atmosphere of 0% CO₂ in air. The tumor cells were routinely subcultured twice weekly by trypsin-EDTA treatment. The cells growing in an exponential growth phase were harvested and counted for tumor inoculation.

2.4.2 Tumor Inoculation

Each mouse was inoculated subcutaneously at the right flank with HT1080 tumor cells (5×10⁶) in 0.2 ml of PBS for tumor development. The animals were randomized and treatment was started when the average tumor volume reached approximately 180 mm³ for the efficacy study. The test article administration and the animal numbers in each group were shown in Table 4.

TABLE 4
Testing Article Formulation Preparation

| | | | Dose | |
|---|---|---|---|---|
| Gr | n | Treatment | (mg/ml) | Formulation |
| 1 | 3 | Vehicle | — | 50 mM Hepes pH 7.0, 20% PEG 400 |
| 2 | 3 | BT66BDC1 | 0.1 | Dilute 100 ul 1 mg/ml BT66BDC1 |
| | | | | (Group4) with 900 ul formulation buffer |
| 3 | 3 | BT66BDC1 | 0.3 | Dilute 300 ul 1 mg/ml BT66BDC1 |
| | | | | (Group4) with 700 ul formulation buffer |
| 4 | 3 | BT66BDC1 | 1.0 | Dilute 70 ul 20 mg/ml BT66BDC1 with |
| | | | | 1330 ul formulation buffer |

2.4.3 Observations

All the procedures related to animal handling, care and the treatment in the study were performed according to the guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured every day), eye/hair matting and any other abnormal effect as stated in the protocol. Death and observed clinical signs were recorded on the basis of the numbers of animals within each subset.

2.4.4 Tumor Measurements and the Endpoints

The major endpoint was to see if the tumor growth could be delayed or mice could be cured. Tumor volume was measured three times weekly in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

2.4.5 Plasma Collection

On PG-D14, mice of group 2, 3 were re-dosed and plasma was collected at 5 min, 15 min, 30 min, 60 min and 120 min after the dosing for PK analysis.

2.4.6 Statistical Analysis

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, comparisons between groups were carried out with Games-Howell test. All data were analyzed using Prism. P<0.05 was considered to be statistically significant.

2.5 Results

2.5.1 Mortality, Morbidity, and Body Weight Gain or Loss

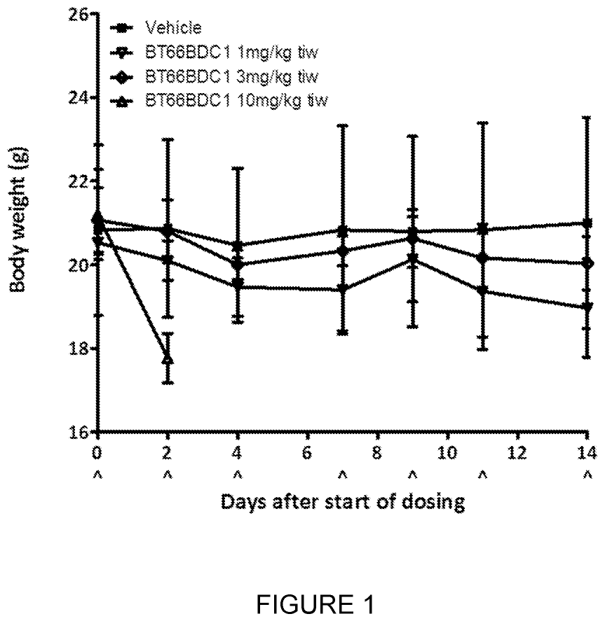

Body weight was monitored regularly as an indirect measure of toxicity. Body weight changes in female Balb/c nude mice bearing HT1080 dosed with BT66BDC1 are shown in FIG. 1. Mice treated with BT66BDC1 at 10 mg/kg showed severe bodyweight loss and death of 3/3 animals.

2.5.2 Tumor Volume Trace

Mean tumor volume over time in female Balb/c nude mice bearing HT1080 xenograft is shown in Table 5.

TABLE 5
Tumor volume trace over time

| | | Days after the start of treatment | | | | | | |
|---|---|---|---|---|---|---|---|---|
| Gr. | Treatment | 0 | 2 | 4 | 7 | 9 | 11 | 14 |
| 1 | Vehicle, | 180 ± 13 | 322 ± 38 | 660 ± 97 | 1156 ± 229 | 1522 ± 367 | 1830 ± 468 | 2232 ± 559 |
| | tiw | | | | | | | |
| 2 | BT66BDC1, | 183 ± 26 | 275 ± 46 | 482 ± 142 | 635 ± 190 | 762 ± 196 | 827 ± 209 | 967 ± 241 |
| | 1 mpk, tiw | | | | | | | |
| 3 | BT66BDC1, | 181 ± 16 | 225 ± 49 | 130 ± 34 | 53 ± 7 | 23 ± 7 | 11 ± 6 | 0 ± 0 |
| | 3 mpk, tiw | | | | | | | |
| 4 | BT66BDC1, | 181 ± 28 | 120 ± 11 | | | | | |
| | 10 mpk, tiw | | | | | | | |

2.5.3 Tumor Growth Curve

The tumor growth curve is shown in FIG. 2.

2.5.4 Tumor Growth Inhibition Analysis

Tumor growth inhibition rate for BT66BDC1 in the HT1080 xenograft model was calculated based on tumor volume measurements at day 14 after the start of treatment.

TABLE 6
Tumor growth inhibition analysis (TIC and TGI)

| | | Tumor | | | |
|---|---|---|---|---|---|
| Gr | Treatment | Volume (mm³)^{a} | T/C^{b} (%) | TGI (%) | P value |
| 1 | Vehicle, tiw | 2232 ± 559 | — | — | — |
| 2 | BT66BDC1, | 967 ± 241 | 43.3 | 61.8 | p > 0.05 |
| | 1 mpk, tiw | | | | |
| 3 | BT66BDC1, | 0 ± 0 | 0.0 | 108.8 | p < 0.01 |
| | 3 mpk, tiw | | | | |
| 4 | BT66BDC1, | — | — | — | — |
| | 10 mpk, tiw | | | | |

^{a}Mean ± SEM.
^{b}Tumor Growth Inhibition is calculated by dividing the group average tumor volume for the treated group by the group average tumor volume for the control group (T/C).

2.6 Results Summary and Discussion

In this study, the therapeutic efficacy of BT66BDC1 in the HT1080 xenograft model was evaluated. The measured body weights and body weight changes are shown in FIG. 1. Tumor volume of all treatment groups at various time points are shown in Tables 5, 6 and FIG. 2.

The mean tumor size of vehicle treated mice reached 2232 mm³ on day 14. BT66BDC1 at 1 mg/kg and 3 mg/kg produced dose-dependent antitumor activity with tumor measured at 967 mm³ (TGI=61.8%, p>0.05) and 0 mm³ (TGI=108.8%, p<0.01) respectively. Among them, BT66BDC1 at 3 mg/kg completely eradicated the tumors by day 14. BT66BDC1 at 10 mg/kg also rapidly regressed the tumor after treatment, but caused severe bodyweight loss and death of 3/3 animals.

3. In Vivo Efficacy Test of BT66BDC1 in Treatment of MOLP-8 Xenograft in CB17-SCID Mice

3.1 Study Objective

The objective of this study was to evaluate the in vivo anti-tumor efficacy of BT66BDC1 in the treatment of the subcutaneous MOLP-8 xenograft model in CB17-SCID mice.

3.2 Experimental Design

TABLE 7

| | | | | Dosing | | |
|---|---|---|---|---|---|---|
| | | | Dosage | Volume | Dosing | |
| Gr | n | Treatment | (mg/kg) | (μl/g) | Route | Schedule |
| 1 | 3 | Vehicle | — | 10 | i.v. | tiw*2 weeks |
| 2 | 3 | BT66BDC1 | 1 | 10 | i.v. | tiw*2 weeks |
| 3 | 3 | BT66BDC1 | 3 | 10 | i.v. | tiw*2 weeks |
| 4 | 3 | BT66BDC1 | 10 | 10 | i.v. | tiw*2 weeks |

Note:
n: animal number; Dosing volume: adjust dosing volume based on body weight 10 μl/g

3.3 Materials

3.3.1 Animals and Housing Condition

3.3.1.1 Animals
Species: Mus Musculus
Strain: CB17-SCID
Age: 6-8 weeks
Sex: female
Body weight: 18-22 g
Number of animals: 18 mice plus spare
Animal supplier: Shanghai SLAC Laboratory Animal Co., LTD.

3.3.1.2 Housing Condition
The mice were kept in individual ventilation cages at constant temperature and humidity with 3 animals in each cage.
   Temperature: 20˜26° C.
   Humidity 40-70%.
Cages: Made of polycarbonate. Size: 300 mm×180 mm×150 mm. The bedding material was corn cob, which was changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, the date received, treatment, study number, group number and the starting date of the treatment.
Animal identification: Animals were marked by ear coding.

3.3.2 Test and Positive Control Articles
Product identification: BT66BDC1
Physical description: DMSO stock
MW: 3234.4, Purity: >95%
Concentration: 20 mg/ml
Package and storage condition: stored at −80° C.

3.4 Experimental Methods and Procedures

3.4.1 Cell Culture

The MOLP-8 tumor cells were maintained in vitro as a monolayer culture in RPMI1640 medium supplemented with 10% heat inactivated fetal bovine serum at 37° C. in an atmosphere of 5% CO₂ in air. The tumor cells were routinely subcultured twice weekly by trypsin-EDTA treatment. The cells growing in an exponential growth phase were harvested and counted for tumor inoculation.

3.4.2 Tumor Inoculation

Each mouse was inoculated subcutaneously at the right flank with MOLP-8 tumor cells (10×10⁶) in 0.2 ml PBS with 50% matrigel for tumor development. The animals were randomized and treatment was started when the average tumor volume reaches approximately 150-200 mm³ for the efficacy study. The test article administration and the animal numbers in each group were shown in Table 8:

TABLE 8
Testing Article Formulation Preparation

| | | | Dose | |
|---|---|---|---|---|
| Gr | n | Treatment | (mg/ml) | Formulation |
| 1 | 3 | Vehicle | — | 50 mM Hepes pH 7.0, 20% PEG 400 |
| 2 | 3 | BT66BDC1 | 0.1 | Dilute 100 μl 1 mg/ml BT66BDC1 |
| | | | | (Group4) with 900 μl formulation buffer |
| 3 | 3 | BT66BDC1 | 0.3 | Dilute 300 μl 1 mg/ml BT66BDC1 |
| | | | | (Group4) with 700 μl formulation buffer |
| 4 | 3 | BT66BDC1 | 1.0 | Dilute 70 μl 20 mg/ml BT66BDC1 with |
| | | | | 1330 μl formulation buffer |

3.4.3 Observations

All the procedures related to animal handling, care and the treatment in the study were performed according to the guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

3.4.4 Tumor Measurements and the Endpoints

The major endpoint was to see if the tumor growth could be delayed or mice could be cured. Tumor size was measured three times weekly in two dimensions using a caliper, and the volume was expressed in mm³ using the formula: V=0.5 a×b² where a and b are the long and short diameters of the tumor, respectively. The tumor size was then used for calculations of T/C value. The T/C value (in percent) is an indication of antitumor effectiveness; T and C are the mean volumes of the treated and control groups, respectively, on a given day.

TGI was calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

3.4.5 Sample Collection

On PG-D14, mice of group 2, 3 were re-dosed and plasma was collected at 5 min, 15 min, 30 min, 60 min and 120 min after the dosing for PK analysis.

3.4.6 Statistical Analysis

Summary statistics, including mean and the standard error of the mean (SEM), are provided for the tumor volume of each group at each time point.

Statistical analysis of difference in tumor volume among the groups was conducted on the data obtained at the best therapeutic time point after the final dose.

A one-way ANOVA was performed to compare tumor volume among groups, and when a significant F-statistics (a ratio of treatment variance to the error variance) was obtained, multiple comparison procedures will be applied after ANOVA. The potential synergistic effect between treatments will be analyzed by two-way ANOVA. All data will be analyzed using SPSS 17.0. p<0.05 is considered to be statistically significant.

3.5 Results

3.5.1 Mortality, Morbidity, and Body Weight Gain or Loss

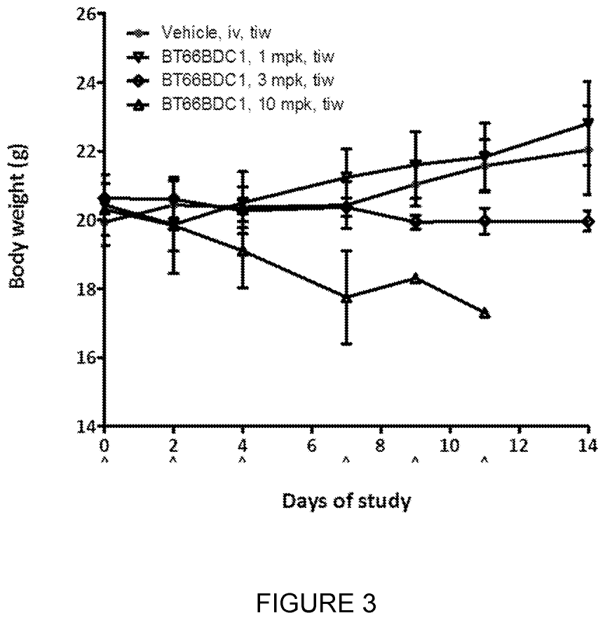

Animal body weight was monitored regularly as an indirect measure of toxicity. Body weight change in female CB17-SCID mice bearing MOLP-8 tumor dosed with BT66BDC1 is shown in FIG. 3 Mice treated with BT66BDC1 at 10 mg/kg showed bodyweight loss and animal death.

3.5.2 Tumor Volume Trace

Mean tumor volume overtime in female CB17-SCID mice bearing MOLP-8 xenograft is shown in Table 9.

TABLE 9
Tumor volume trace over time

| | | Days after the start of treatment | | | | | | |
|---|---|---|---|---|---|---|---|---|
| Group | Treatment | 0 | 2 | 4 | 7 | 9 | 11 | 14 |
| 1 | Vehicle, | 167 ± 24 | 286 ± 52 | 429 ± 77 | 713 ± 101 | 948 ± 126 | 1242 ± 223 | 1771 ± 242 |
| | iv, biw | | | | | | | |
| 2 | BT66BDC1, | 167 ± 20 | 250 ± 59 | 357 ± 76 | 542 ± 47 | 706 ± 57 | 841 ± 74 | 1114 ± 59 |
| | 1 mpk, | | | | | | | |
| | iv, tiw | | | | | | | |
| 3 | BT66BDC1, | 166 ± 12 | 276 ± 60 | 187 ± 39 | 144 ± 18 | 116 ± 23 | 95 ± 15 | 77 ± 14 |
| | 3 mpk, | | | | | | | |
| | iv, tiw | | | | | | | |
| 4 | BT66BDC1, | 166 ± 13 | 186 ± 22 | 70 ± 5 | 33 ± 1 | 30 | 15 | — |
| | 10 mpk, | | | | | | | |
| | iv, tiw | | | | | | | |

3.5.3 Tumor Growth Curve

The tumor growth curve is shown in FIG. 4.

3.5.4 Tumor Growth Inhibition Analysis

Tumor growth inhibition rate for BT66BDC1 in the MOLP-8 xenograft model was calculated based on tumor volume measurements at day 14 after the start of treatment.

TABLE 10
Tumor growth inhibition analysis (TIC and TGI)

| | | Tumor Volume | | | |
|---|---|---|---|---|---|
| Gr | Treatment | (mm³)^{a} | T/C^{b} (%) | TGI (%) | P value |
| 1 | Vehicle, | 1771 ± 242 | — | — | — |
| | iv, biw | | | | |
| 2 | BT66BDC1, | 1114 ± 59 | 63 | 41 | p < 0.05 |
| | 1 mpk, iv, biw | | | | |
| 3 | BT66BDC1, | 77 ± 14 | 4 | 106 | p < 0.001 |
| | 3 mpk, iv, biw | | | | |
| 4 | BT66BDC1, | — | — | — | — |
| | 10 mpk, iv, biw | | | | |

^{a}Mean ± SEM.
^{b}Tumor Growth Inhibition is calculated by dividing the group average tumor volume for the treated group by the group average tumor volume for the control group (T/C).

3.6 Results Summary and Discussion

In this study, the therapeutic efficacy of BT66BDC1 in the MOLP-8 xenograft model was evaluated. The measured body weights and body weight changes are shown in FIG. 3. Tumor volumes of all treatment groups at various time points are shown in Tables 9, 10 and FIG. 4.

The mean tumor size of vehicle treated mice reached 1771 mm³ on day 14. BT66BDC1 at 1 mg/kg and 3 mg/kg produced dose-dependent antitumor activity with tumor measured at 1114 mm³ (TGI=41%, p<0.05) and 77 mm³ (TGI=106%, p<0.001) respectively. BT66BDC1 at 10 mg/kg rapidly regressed the tumor after treatment, but caused bodyweight loss and death of 3/3 animals.

1. A peptide ligand specific for CD38 comprising a polypeptide comprising at least three cysteine residues, separated by at least two loop sequences, and an aromatic molecular scaffold which forms covalent bonds with the cysteine residues of the polypeptide such that at least two polypeptide loops are formed on the molecular scaffold.

2. The peptide ligand as defined in claim 1, wherein said loop sequences comprise 2, 3, 5, 6 or 7 amino acids.

3. The peptide ligand as defined in claim 1, wherein the polypeptide comprises an amino acid sequence selected from:
(SEQ ID NO: 87)
Cᵢ-F-X₁-L-D-X₂-X₃-Cᵢᵢ-F-D-X₄-X₅-X₆-X₇-Cᵢᵢᵢ;
(SEQ ID NO: 88)
Cᵢ-I-R/N-Y-G/A-D/N-I-Cᵢᵢ-X₁-D/H-P/T-D/E-X₂-X₃-
Cᵢᵢᵢ;
(SEQ ID NO: 89)
Cᵢ-F-X₁-L-D-G-E-Cᵢᵢ-F-X₂-X₃-G/P-X₄-X₅-Cᵢᵢᵢ;
(SEQ ID NO: 2)
CᵢVNFGSVCᵢᵢWDPDSRCᵢᵢᵢ;
(SEQ ID NO: 90)
Cᵢ-X₁-X₂-Cᵢᵢ-A-D-F/M-P-I-X₃-X₄-Cᵢᵢᵢ;
(SEQ ID NO: 74)
CᵢDYCᵢᵢVRLGLTGCᵢᵢᵢ;
(SEQ ID NO: 75)
CᵢGWCᵢᵢSDQIDGFCᵢᵢᵢ;
(SEQ ID NO: 76)
CᵢAWCᵢᵢSDPIDGFCᵢᵢᵢ;
(SEQ ID NO: 77)
CᵢDWCᵢᵢIDPGVSFCᵢᵢᵢ;
(SEQ ID NO: 78)
CᵢSWCᵢᵢVDDGLPFCᵢᵢᵢ;
(SEQ ID NO: 79)
CᵢTWCᵢᵢVDDGLSFCᵢᵢᵢ;
(SEQ ID NO: 80)
CᵢTWCᵢᵢVDDETWNCᵢᵢᵢ;
(SEQ ID NO: 81)
CᵢDYCᵢᵢIRLGLTGCᵢᵢᵢ;
(SEQ ID NO: 82)
CᵢDWCᵢᵢTDNIPGICᵢᵢᵢ;
(SEQ ID NO: 91)
Cᵢ-A/N-W/F-L-Cᵢᵢ-P/D-N/D-L-Cᵢᵢᵢ;
(SEQ ID NO: 92)
Cᵢ-D-F-T-M-P-Cᵢᵢ-X₁-X₂-W-X₃-X₄-Cᵢᵢᵢ;
and
(SEQ ID NO: 28)
CᵢIFDYDCᵢᵢDAWSACᵢᵢᵢ;
wherein X₁-X₆ represent any amino acid residue, X₇ is either absent or represents any amino acid, and Cᵢ, Cᵢᵢ and Cᵢᵢᵢ represent first, second and third cysteine residues, respectively or a pharmaceutically acceptable salt thereof.

4. The peptide ligand as defined in claim 1, wherein said polypeptide comprises three cysteine residues separated by two loop sequences both of which consist of 6 amino acids or one of which consists of 5 amino acids and the other of which consists of 6 amino acids, and said polypeptide comprises an amino acid sequence selected from:
(SEQ ID NO: 87)
Cᵢ-F-X₁-L-D-X₂-X₃₋Cᵢᵢ-F-D-X₄-X₅-X₆-X₇-Cᵢᵢᵢ;
(SEQ ID NO: 88)
Cᵢ-I-R/N-Y-G/A-D/N-I-Cᵢᵢ-X₁-D/H-P/T-D/E-X₂-X₃-
Cᵢᵢᵢ;
(SEQ ID NO: 89)
Cᵢ-F-X₁-L-D-G-E-Cᵢᵢ-F-X₂-X₃-G/P-X₄-X₅-Cᵢᵢᵢ;
and
(SEQ ID NO: 2)
CᵢVNFGSVCᵢᵢWDPDSRCᵢᵢᵢ;
wherein X₁-X₆ represent any amino acid residue, X₇ is either absent or represents any amino acid, and Cᵢ, Cᵢᵢ and Cᵢᵢᵢ represent first, second and third cysteine residues, respectively or a pharmaceutically acceptable salt thereof.

5. The peptide ligand as defined in claim 1, wherein said polypeptide comprises three cysteine residues separated by two loop sequences the first of which consists of 2 amino acids and the second of which consists of 7 amino acids, and said polypeptide comprises an amino acid sequence selected from:
(SEQ ID NO: 90)
Cᵢ-X₁-X₂-Cᵢᵢ-A-D-F/M-P-I-X₃-X₄-Cᵢᵢᵢ;
(SEQ ID NO: 74)
CᵢDYCᵢᵢVRLGLTGCᵢᵢᵢ;
(SEQ ID NO: 75)
CᵢGWCᵢᵢSDQIDGFCᵢᵢᵢ;
(SEQ ID NO: 76)
CᵢAWCᵢᵢSDPIDGFCᵢᵢᵢ;
(SEQ ID NO: 77)
CᵢDWCᵢᵢIDPGVSFCᵢᵢᵢ;
(SEQ ID NO: 78)
CᵢSWCᵢᵢVDDGLPFCᵢᵢᵢ;
(SEQ ID NO: 79)
CᵢTWCᵢᵢVDDGLSFCᵢᵢᵢ;
(SEQ ID NO: 80)
CᵢTWCᵢᵢVDDETWNCᵢᵢᵢ;
(SEQ ID NO: 81)
CᵢDYCᵢᵢIRLGLTGCᵢᵢᵢ;
and
(SEQ ID NO: 82)
CᵢDWCᵢᵢTDNIPGICᵢᵢᵢ;
wherein X₁-X₄ represent any amino acid residue, and Cᵢ, Cᵢᵢ and Cᵢᵢᵢ represent first, second and third cysteine residues, respectively or a pharmaceutically acceptable salt thereof.

6. The peptide ligand as defined in claim 1, wherein said polypeptide comprises three cysteine residues separated by two loop sequences both of which consist of 3 amino acids, and said polypeptide comprises an amino acid sequence:
(SEQ ID NO: 91)
Cᵢ-A/N-W/F-L-Cᵢᵢ-P/D-N/D-L-Cᵢᵢᵢ;
wherein Cᵢ, Cᵢᵢ and Cᵢᵢᵢ represent first, second and third cysteine residues, respectively or a pharmaceutically acceptable salt thereof.

7. The peptide ligand as defined in claim 1, wherein said polypeptide comprises three cysteine residues separated by two loop sequences both of which consist of 5 amino acids, and said polypeptide comprises an amino acid sequence selected from:
(SEQ ID NO: 92)
Cᵢ-D-F-T-M-P-Cᵢᵢ-X₁-X₂-W-X₃-X₄-Cᵢᵢᵢ;
and
(SEQ ID NO: 28)
CᵢIFDYDCᵢᵢDAWSACᵢᵢᵢ;
wherein X₁-X₄ represent any amino acid residue, and Cᵢ, Cᵢᵢ and Cᵢᵢᵢ represent first, second and third cysteine residues, respectively or a pharmaceutically acceptable salt thereof.

8. The peptide ligand as defined in claim 3, wherein the amino acid sequence Cᵢ—F—X₁-L-D-X₂—X₃—Cᵢᵢ—F-D-X₄—X₅—X₆—X₇—Cᵢᵢᵢ (SEQ ID NO: 87) is selected from:
(SEQ ID NO: 1)
CᵢFELDGTCᵢᵢFDWAQECᵢᵢᵢ;
(SEQ ID NO: 29)
CᵢFWLDGECᵢᵢFDWNHECᵢᵢᵢ;
(SEQ ID NO: 30)
CᵢFHLDGECᵢᵢFDLENTCᵢᵢᵢ;
(SEQ ID NO: 32)
CᵢFSLDGECᵢᵢFDLSGECᵢᵢᵢ;
(SEQ ID NO: 33)
CᵢFTLDGECᵢᵢFDWTHECᵢᵢᵢ;
(SEQ ID NO: 34)
CᵢFKLDGVCᵢᵢFDLFHECᵢᵢᵢ;
(SEQ ID NO: 35)
CᵢFMLDGECᵢᵢFDLNKECᵢᵢᵢ;
(SEQ ID NO: 36)
CᵢFKLDGECᵢᵢFDWTHECᵢᵢᵢ;
(SEQ ID NO: 37)
CᵢFTLDGECᵢᵢFDWDAECᵢᵢᵢ;
(SEQ ID NO: 38)
CᵢFELDGSCᵢᵢFDFDHECᵢᵢᵢ;
(SEQ ID NO: 39)
CᵢFTLDGECᵢᵢFDVNRECᵢᵢᵢ;
(SEQ ID NO: 40)
CᵢFWLDHECᵢᵢFDWTHECᵢᵢᵢ;
(SEQ ID NO: 41)
CᵢFQLDGECᵢᵢFDIYRECᵢᵢᵢ;
(SEQ ID NO: 42)
CᵢFELDGNCᵢᵢFDWTHECᵢᵢᵢ;
(SEQ ID NO: 43)
CᵢFHLDGECᵢᵢFDYEHECᵢᵢᵢ;
(SEQ ID NO: 44)
CᵢFSLDGECᵢᵢFDIASECᵢᵢᵢ;
(SEQ ID NO: 45)
CᵢFQLDGECᵢᵢFDTSHECᵢᵢᵢ;
(SEQ ID NO: 46)
CᵢFSLDGACᵢᵢFDWTHECᵢᵢᵢ;
(SEQ ID NO: 47)
CᵢFVLDGECᵢᵢFDYYEECᵢᵢᵢ;
(SEQ ID NO: 48)
CᵢFRLDDECᵢᵢFDWTHECᵢᵢᵢ;
(SEQ ID NO: 49)
CᵢFRLDGVCᵢᵢFDLDDECᵢᵢᵢ;
(SEQ ID NO: 50)
CᵢFRLDGECᵢᵢFDMGQECᵢᵢᵢ;
(SEQ ID NO: 51)
CᵢFTLDGACᵢᵢFDLDGECᵢᵢᵢ;
(SEQ ID NO: 52)
CᵢFTLDGQCᵢᵢFDWTHECᵢᵢᵢ;
(SEQ ID NO: 53)
CᵢFLLDGECᵢᵢFDWMQECᵢᵢᵢ;
(SEQ ID NO: 54)
CᵢFELDGDCᵢᵢFDWTHECᵢᵢᵢ;
(SEQ ID NO: 55)
CᵢFTLDGTCᵢᵢFDWTHECᵢᵢᵢ;
(SEQ ID NO: 56)
CᵢFHLDGVCᵢᵢFDWTHECᵢᵢᵢ;
(SEQ ID NO: 57)
CᵢFYLDGTCᵢᵢFDWTHECᵢᵢᵢ;
(SEQ ID NO: 58)
CᵢFLLDGECᵢᵢFDWAQECᵢᵢᵢ;
(SEQ ID NO: 59)
CᵢFHLDGECᵢᵢFDLAKTCᵢᵢᵢ;
(SEQ ID NO: 62)
CᵢFTLDGECᵢᵢFDLDGWCᵢᵢᵢ;
(SEQ ID NO: 63)
CᵢFLLDGECᵢᵢFDLIGECᵢᵢᵢ;
(SEQ ID NO: 67)
CᵢFWLDGECᵢᵢFDLGGQCᵢᵢᵢ;
(SEQ ID NO: 68)
CᵢFELDGECᵢᵢFDLDNQCᵢᵢᵢ;
(SEQ ID NO: 69)
CᵢFWLDGECᵢᵢFDLYGGCᵢᵢᵢ;
(SEQ ID NO: 70)
CᵢFRLDGECᵢᵢFDISNECᵢᵢᵢ;
(SEQ ID NO: 72)
CᵢFWLDGECᵢᵢFDFGGCᵢᵢᵢ;
and
(SEQ ID NO: 73)
CᵢFTLDGACᵢᵢFDWTHECᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
A-(SEQ ID NO: 1)-A (herein referred to as 66-01-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K (herein referred to as 66-08-01-N005);
Ac-(SEQ ID NO: 29)-A-Sar₆-K(DOTA) (herein referred to as 66-08-01-N016);
A-(SEQ ID NO: 30)-A (herein referred to as 66-08-44-N001);
Ac-A-(SEQ ID NO: 30)-A-Sar₆-K(Biot) (herein referred to as 66-08-01-N003);
A-(SEQ ID NO: 32)-A (herein referred to as 66-08-02-N001);
A-(SEQ ID NO: 33)-A (herein referred to as 66-08-03-N001);
A-(SEQ ID NO: 34)-A (herein referred to as 66-08-04-N001);
Ac-A-(SEQ ID NO: 35)-A (herein referred to as 66-08-05-N001);
A-(SEQ ID NO: 36)-A (herein referred to as 66-08-06-N001);
A-(SEQ ID NO: 37)-A (herein referred to as 66-08-07-N001);
A-(SEQ ID NO: 38)-A (herein referred to as 66-08-09-N001);
A-(SEQ ID NO: 39)-A (herein referred to as 66-08-13-N001);
A-(SEQ ID NO: 40)-A (herein referred to as 66-08-15-N001);
A-(SEQ ID NO: 41)-A (herein referred to as 66-08-17-N001);
A-(SEQ ID NO: 42)-A (herein referred to as 66-08-18-N001);
A-(SEQ ID NO: 43)-A (herein referred to as 66-08-20-N001);
A-(SEQ ID NO: 44)-A (herein referred to as 66-08-22-N001);
A-(SEQ ID NO: 45)-A (herein referred to as 66-08-24-N001);
A-(SEQ ID NO: 46)-A (herein referred to as 66-08-26-N001);
A-(SEQ ID NO: 47)-A (herein referred to as 66-08-27-N001);
A-(SEQ ID NO: 48)-A (herein referred to as 66-08-28-N001);
A-(SEQ ID NO: 49)-A (herein referred to as 66-08-29-N001);
A-(SEQ ID NO: 50)-A (herein referred to as 66-08-30-N001);
A-(SEQ ID NO: 51)-A (herein referred to as 66-08-31-N001);
A-(SEQ ID NO: 52)-A (herein referred to as 66-08-32-N001);
A-(SEQ ID NO: 53)-A (herein referred to as 66-08-33-N001);
A-(SEQ ID NO: 54)-A (herein referred to as 66-08-34-N001);
A-(SEQ ID NO: 55)-A (herein referred to as 66-08-35-N001);
A-(SEQ ID NO: 56)-A (herein referred to as 66-08-36-N001);
A-(SEQ ID NO: 57)-A (herein referred to as 66-08-41-N001);
A-(SEQ ID NO: 58)-A (herein referred to as 66-08-43-N001);
A-(SEQ ID NO: 59)-A (herein referred to as 66-08-45-N001);
A-(SEQ ID NO: 62)-A (herein referred to as 66-08-48-N001);
A-(SEQ ID NO: 63)-A (herein referred to as 66-08-49-N001);
A-(SEQ ID NO: 67)-A (herein referred to as 66-08-53-N001);
A-(SEQ ID NO: 68)-A (herein referred to as 66-08-54-N001);
A-(SEQ ID NO: 69)-A (herein referred to as 66-08-55-N001);
A-(SEQ ID NO: 70)-A (herein referred to as 66-08-56-N001);
A-(SEQ ID NO: 72)-A (herein referred to as 66-08-58-N001); and
A-(SEQ ID NO: 73)-A (herein referred to as 66-08-N002).

9. The peptide ligand as defined in claim 3, wherein the amino acid sequence Cᵢ—I—R/N—Y-G/A-D/N—I—Cᵢᵢ—X₁-D/H—P/T-D/E-X₂—X₃—Cᵢᵢᵢ (SEQ ID NO: 88) is selected from:
(SEQ ID NO: 83)
CᵢIRYGDICᵢᵢYDPDHSCᵢᵢᵢ;
(SEQ ID NO: 84)
CᵢIRYGDICᵢᵢFHPDYTCᵢᵢᵢ;
and
(SEQ ID NO: 85)
CᵢINYANICᵢᵢLDTEKMCᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
A-(SEQ ID NO: 83)-A (herein referred to as 66-17-01-N001);
A-(SEQ ID NO: 84)-A (herein referred to as 66-17-N001); and
A-(SEQ ID NO: 85)-A (herein referred to as 66-18-N001).

10. The peptide ligand as defined in claim 3, wherein the amino acid sequence Cᵢ—F—X₁-L-D-G-E-Cᵢᵢ—F—X₂—X₃-G/P—X₄—X₅—Cᵢᵢᵢ (SEQ ID NO: 89) is selected from:
(SEQ ID NO: 60)
CᵢFELDGECᵢᵢFEIFGEPCᵢᵢᵢ;
(SEQ ID NO: 61)
CᵢFVLDGECᵢᵢFEIGERCᵢᵢᵢ;
(SEQ ID NO: 64)
CᵢFELDGECᵢᵢFSFPGTCᵢᵢᵢ;
(SEQ ID NO: 65)
CᵢFELDGECᵢᵢFSWPYPCᵢᵢᵢ;
(SEQ ID NO: 66)
CᵢFTLDGECᵢᵢFLLGENCᵢᵢᵢ;
and
(SEQ ID NO: 71)
CᵢFELDGECᵢᵢFNIGSKCᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
A-(SEQ ID NO: 60)-A (herein referred to as 66-08-46-N001);
A-(SEQ ID NO: 61)-A (herein referred to as 66-08-47-N001);
A-(SEQ ID NO: 64)-A (herein referred to as 66-08-50-N001);
A-(SEQ ID NO: 65)-A (herein referred to as 66-08-51-N001);
A-(SEQ ID NO: 66)-A (herein referred to as 66-08-52-N001); and
A-(SEQ ID NO: 71)-A (herein referred to as 66-08-57-N001).

11. The peptide ligand as defined in claim 3, wherein the polypeptide comprises an amino acid sequence:
A-(SEQ ID NO: 2)-A (herein referred to as 66-02-N002).

12. The peptide ligand as defined in claim 3, wherein the amino acid sequence Cᵢ—X₁—X₂-A-D-F/M-Cᵢᵢ—P—I—X₃—X₄—Cᵢᵢᵢ (SEQ ID NO: 90) is selected from:
(SEQ ID NO: 3)
CᵢVPCᵢᵢADFPIWYCᵢᵢᵢ;
(SEQ ID NO: 4)
CᵢVPCᵢᵢADFPIWWCᵢᵢᵢ;
(SEQ ID NO: 5)
CᵢTPCᵢᵢADFPIWSCᵢᵢᵢ;
(SEQ ID NO: 6)
CᵢTPCᵢᵢADFPIHTCᵢᵢᵢ;
(SEQ ID NO: 7)
CᵢVHCᵢᵢADFPIWGCᵢᵢᵢ;
(SEQ ID NO: 8)
CᵢVPCᵢᵢADFPIWGCᵢᵢᵢ;
(SEQ ID NO: 9)
CᵢVMCᵢᵢADFPIWGCᵢᵢᵢ;
(SEQ ID NO: 10)
CᵢTPCᵢᵢADFPIWYCᵢᵢᵢ;
(SEQ ID NO: 11)
CᵢTPCᵢᵢADFPILTCᵢᵢᵢ;
(SEQ ID NO: 12)
CᵢVACᵢᵢADFPIWGCᵢᵢᵢ;
(SEQ ID NO: 13)
CᵢTPCᵢᵢADFPIYGCᵢᵢᵢ;
(SEQ ID NO: 14)
CᵢTPCᵢᵢADFPILDCᵢᵢᵢ;
(SEQ ID NO: 15)
CᵢVKCᵢᵢADFPIWGCᵢᵢᵢ;
(SEQ ID NO: 16)
CᵢTPCᵢᵢADMPIWTCᵢᵢᵢ;
(SEQ ID NO: 17)
CᵢIPCᵢᵢADFPIWGCᵢᵢᵢ;
(SEQ ID NO: 18)
CᵢIPCᵢᵢADFPISVCᵢᵢᵢ;
(SEQ ID NO: 19)
CᵢVPCᵢᵢADFPISFCᵢᵢᵢ;
(SEQ ID NO: 20)
CᵢIPCᵢᵢADFPISFCᵢᵢᵢ;
(SEQ ID NO: 21)
CᵢVPCᵢᵢADFPISVCᵢᵢᵢ;
(SEQ ID NO: 22)
CᵢVPCᵢᵢADFPIFTCᵢᵢᵢ;
(SEQ ID NO: 23)
CᵢIPCᵢᵢADFPIFTCᵢᵢᵢ;
and
(SEQ ID NO: 24)
CᵢTPCᵢᵢADFPIWGCᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 4)-A (herein referred to as 66-03-00-N005);
A-(SEQ ID NO: 5)-A (herein referred to as 66-03-01-N001);
A-(SEQ ID NO: 6)-A (herein referred to as 66-03-02-N001);
A-(SEQ ID NO: 7)-A (herein referred to as 66-03-03-N001);
A-(SEQ ID NO: 8)-A (herein referred to as 66-03-04-N001);
Ac-(SEQ ID NO: 8) (herein referred to as 66-03-04-N002);
A-(SEQ ID NO: 9)-A (herein referred to as 66-03-05-N001);
A-(SEQ ID NO: 10)-A (herein referred to as 66-03-06-N001);
Ac-(SEQ ID NO: 10) (herein referred to as 66-03-06-N002);
A-(SEQ ID NO: 11)-A (herein referred to as 66-03-07-N001);
A-(SEQ ID NO: 12)-A (herein referred to as 66-03-08-N001);
A-(SEQ ID NO: 13)-A (herein referred to as 66-03-09-N001);
A-(SEQ ID NO: 14)-A (herein referred to as 66-03-10-N001);
A-(SEQ ID NO: 15)-A (herein referred to as 66-03-11-N001);
Ac-(SEQ ID NO: 15) (herein referred to as 66-03-11-N002);
A-(SEQ ID NO: 16)-A (herein referred to as 66-03-15-N001);
A-(SEQ ID NO: 17)-A (herein referred to as 66-03-16-N001);
A-(SEQ ID NO: 18)-A (herein referred to as 66-03-24-N003);
A-(SEQ ID NO: 19)-A (herein referred to as 66-03-25-N002);
A-(SEQ ID NO: 20)-A (herein referred to as 66-03-26-N003);
A-(SEQ ID NO: 21)-A (herein referred to as 66-03-27-N003);
A-(SEQ ID NO: 22)-A (herein referred to as 66-03-28-N002);
A-(SEQ ID NO: 23)-A (herein referred to as 66-03-29-N003);
A-(SEQ ID NO: 24)-A (herein referred to as 66-03-N002);
A-(SEQ ID NO: 24)-A-Sar₆-K(Biot) (herein referred to as 66-03-N003);
A-(SEQ ID NO: 74)-A (herein referred to as 66-09-N001);
A-(SEQ ID NO: 75)-A-Sar₆-K(Biotin) (herein referred to as 66-10-01-N001);
A-(SEQ ID NO: 76)-A-Sar₆-K(Biotin) (herein referred to as 66-10-02-N001);
A-(SEQ ID NO: 77)-A-Sar₆-K(Biotin) (herein referred to as 66-10-03-N001);
A-(SEQ ID NO: 78)-A-Sar₆-K(Biotin) (herein referred to as 66-10-04-N001);
A-(SEQ ID NO: 79)-A (herein referred to as 66-10-N001);
A-(SEQ ID NO: 80)-A (herein referred to as 66-11-N001);
A-(SEQ ID NO: 81)-A (herein referred to as 66-12-N001); and
A-(SEQ ID NO: 82)-A (herein referred to as 66-13-N001).

13. The peptide ligand as defined in claim 3, wherein the amino acid sequence Cᵢ-A/N—W/F-L-Cᵢᵢ—P/D-N/D-L-Cᵢᵢᵢ (SEQ ID NO: 91) is selected from:
(SEQ ID NO: 31)
CᵢAWLCᵢᵢPNLCᵢᵢᵢ;
and
(SEQ ID NO: 86)
CᵢNFLCᵢᵢDDLCᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
SSQHG-(SEQ ID NO: 31)-A-Sar₆-K (herein referred to as 66-08-01-N006); and
RHSNY-(SEQ ID NO: 86)-A-Sar₆-K(Biotin) (herein referred to as 66-20-00-T001-N001).

14. The peptide ligand as defined in claim 3, wherein the amino acid sequence Cᵢ-D-F-T-M-P—Cᵢᵢ—X₁—X₂—W—X₃—X₄—Cᵢᵢᵢ (SEQ ID NO: 92) is selected from:
(SEQ ID NO: 25)
CᵢDFTMPCᵢᵢENAVKYCᵢᵢᵢ;
(SEQ ID NO: 26)
CᵢDFTMPCᵢᵢPNWNACᵢᵢᵢ;
and
(SEQ ID NO: 27)
CᵢDFTMPCᵢᵢQMWEQCᵢᵢᵢ;
wherein Cᵢ, Cᵢᵢ and Cᵢᵢᵢ represent first, second and third cysteine residues, respectively, or a pharmaceutically acceptable salt thereof,
such as:
the peptide ligand wherein the polypeptide has an amino acid sequence selected from:
A-(SEQ ID NO: 25)-A (herein referred to as 66-05-07-N001);
A-(SEQ ID NO: 26)-A (herein referred to as 66-05-09-N001);
A-(SEQ ID NO: 27)-A (herein referred to as 66-05-N002); and
A-(SEQ ID NO: 28)-A (herein referred to as 66-06-N002).

15. The peptide ligand as defined in claim 1, wherein the molecular scaffold is 1,3,5-tris(bromomethyl)benzene (TBMB) and the polypeptide comprises an amino acid sequence selected from:
A-(SEQ ID NO: 1)-A (herein referred to as 66-01-N002);
A-(SEQ ID NO: 2)-A (herein referred to as 66-02-N002);
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 4)-A (herein referred to as 66-03-00-N005);
A-(SEQ ID NO: 5)-A (herein referred to as 66-03-01-N001);
A-(SEQ ID NO: 6)-A (herein referred to as 66-03-02-N001);
A-(SEQ ID NO: 7)-A (herein referred to as 66-03-03-N001);
A-(SEQ ID NO: 8)-A (herein referred to as 66-03-04-N001);
Ac-(SEQ ID NO: 8) (herein referred to as 66-03-04-N002);
A-(SEQ ID NO: 9)-A (herein referred to as 66-03-05-N001);
A-(SEQ ID NO: 10)-A (herein referred to as 66-03-06-N001);
Ac-(SEQ ID NO: 10) (herein referred to as 66-03-06-N002);
A-(SEQ ID NO: 11)-A (herein referred to as 66-03-07-N001);
A-(SEQ ID NO: 12)-A (herein referred to as 66-03-08-N001);
A-(SEQ ID NO: 13)-A (herein referred to as 66-03-09-N001);
A-(SEQ ID NO: 14)-A (herein referred to as 66-03-10-N001);
A-(SEQ ID NO: 15)-A (herein referred to as 66-03-11-N001);
Ac-(SEQ ID NO: 15) (herein referred to as 66-03-11-N002);
A-(SEQ ID NO: 16)-A (herein referred to as 66-03-15-N001);
A-(SEQ ID NO: 17)-A (herein referred to as 66-03-16-N001);
A-(SEQ ID NO: 18)-A (herein referred to as 66-03-24-N003);
A-(SEQ ID NO: 19)-A (herein referred to as 66-03-25-N002);
A-(SEQ ID NO: 20)-A (herein referred to as 66-03-26-N003);
A-(SEQ ID NO: 21)-A (herein referred to as 66-03-27-N003);
A-(SEQ ID NO: 22)-A (herein referred to as 66-03-28-N002);
A-(SEQ ID NO: 23)-A (herein referred to as 66-03-29-N003);
A-(SEQ ID NO: 24)-A (herein referred to as 66-03-N002);
A-(SEQ ID NO: 24)-A-Sar₆-K(Biot) (herein referred to as 66-03-N003);
A-(SEQ ID NO: 25)-A (herein referred to as 66-05-07-N001);
A-(SEQ ID NO: 26)-A (herein referred to as 66-05-09-N001);
A-(SEQ ID NO: 27)-A (herein referred to as 66-05-N002);
A-(SEQ ID NO: 28)-A (herein referred to as 66-06-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K (herein referred to as 66-08-01-N005);
Ac-(SEQ ID NO: 29)-A-Sar₆-K(DOTA) (herein referred to as 66-08-01-N016);
Ac-A-(SEQ ID NO: 30)-A-Sar₆-K(Biot) (herein referred to as 66-08-01-N003);
A-(SEQ ID NO: 30)-A (herein referred to as 66-08-44-N001);
SSQHG-(SEQ ID NO: 31)-A-Sar₆-K (herein referred to as 66-08-01-N006);
A-(SEQ ID NO: 32)-A (herein referred to as 66-08-02-N001);
A-(SEQ ID NO: 33)-A (herein referred to as 66-08-03-N001);
A-(SEQ ID NO: 34)-A (herein referred to as 66-08-04-N001);
Ac-A-(SEQ ID NO: 35)-A (herein referred to as 66-08-05-N001);
A-(SEQ ID NO: 36)-A (herein referred to as 66-08-06-N001);
A-(SEQ ID NO: 37)-A (herein referred to as 66-08-07-N001);
A-(SEQ ID NO: 38)-A (herein referred to as 66-08-09-N001);
A-(SEQ ID NO: 39)-A (herein referred to as 66-08-13-N001);
A-(SEQ ID NO: 40)-A (herein referred to as 66-08-15-N001);
A-(SEQ ID NO: 41)-A (herein referred to as 66-08-17-N001);
A-(SEQ ID NO: 42)-A (herein referred to as 66-08-18-N001);
A-(SEQ ID NO: 43)-A (herein referred to as 66-08-20-N001);
A-(SEQ ID NO: 44)-A (herein referred to as 66-08-22-N001);
A-(SEQ ID NO: 45)-A (herein referred to as 66-08-24-N001);
A-(SEQ ID NO: 46)-A (herein referred to as 66-08-26-N001);
A-(SEQ ID NO: 47)-A (herein referred to as 66-08-27-N001);
A-(SEQ ID NO: 48)-A (herein referred to as 66-08-28-N001);
A-(SEQ ID NO: 49)-A (herein referred to as 66-08-29-N001);
A-(SEQ ID NO: 50)-A (herein referred to as 66-08-30-N001);
A-(SEQ ID NO: 51)-A (herein referred to as 66-08-31-N001);
A-(SEQ ID NO: 52)-A (herein referred to as 66-08-32-N001);
A-(SEQ ID NO: 53)-A (herein referred to as 66-08-33-N001);
A-(SEQ ID NO: 54)-A (herein referred to as 66-08-34-N001);
A-(SEQ ID NO: 55)-A (herein referred to as 66-08-35-N001);
A-(SEQ ID NO: 56)-A (herein referred to as 66-08-36-N001);
A-(SEQ ID NO: 57)-A (herein referred to as 66-08-41-N001);
A-(SEQ ID NO: 58)-A (herein referred to as 66-08-43-N001);
A-(SEQ ID NO: 59)-A (herein referred to as 66-08-45-N001);
A-(SEQ ID NO: 60)-A (herein referred to as 66-08-46-N001);
A-(SEQ ID NO: 61)-A (herein referred to as 66-08-47-N001);
A-(SEQ ID NO: 62)-A (herein referred to as 66-08-48-N001);
A-(SEQ ID NO: 63)-A (herein referred to as 66-08-49-N001);
A-(SEQ ID NO: 64)-A (herein referred to as 66-08-50-N001);
A-(SEQ ID NO: 65)-A (herein referred to as 66-08-51-N001);
A-(SEQ ID NO: 66)-A (herein referred to as 66-08-52-N001);
A-(SEQ ID NO: 67)-A (herein referred to as 66-08-53-N001);
A-(SEQ ID NO: 68)-A (herein referred to as 66-08-54-N001);
A-(SEQ ID NO: 69)-A (herein referred to as 66-08-55-N001);
A-(SEQ ID NO: 70)-A (herein referred to as 66-08-56-N001);
A-(SEQ ID NO: 71)-A (herein referred to as 66-08-57-N001);
A-(SEQ ID NO: 72)-A (herein referred to as 66-08-58-N001);
A-(SEQ ID NO: 73)-A (herein referred to as 66-08-N002);
A-(SEQ ID NO: 74)-A (herein referred to as 66-09-N001);
A-(SEQ ID NO: 75)-A-Sar₆-K(Biotin) (herein referred to as 66-10-01-N001);
A-(SEQ ID NO: 76)-A-Sar₆-K(Biotin) (herein referred to as 66-10-02-N001);
A-(SEQ ID NO: 77)-A-Sar₆-K(Biotin) (herein referred to as 66-10-03-N001);
A-(SEQ ID NO: 78)-A-Sar₆-K(Biotin) (herein referred to as 66-10-04-N001);
A-(SEQ ID NO: 79)-A (herein referred to as 66-10-N001);
A-(SEQ ID NO: 80)-A (herein referred to as 66-11-N001);
A-(SEQ ID NO: 81)-A (herein referred to as 66-12-N001);
A-(SEQ ID NO: 82)-A (herein referred to as 66-13-N001);
A-(SEQ ID NO: 83)-A (herein referred to as 66-17-01-N001);
A-(SEQ ID NO: 84)-A (herein referred to as 66-17-N001);
A-(SEQ ID NO: 85)-A (herein referred to as 66-18-N001); and
RHSNY-(SEQ ID NO: 86)-A-Sar₆-K(Biotin) (herein referred to as 66-20-00-T001-N001),
such as:
the peptide ligand wherein the polypeptide has an amino acid sequence selected from:
A-(SEQ ID NO: 3)-A (herein referred to as 66-03-00-N004);
(β-Ala)-Sar₁₀-A-(SEQ ID NO: 3) (herein referred to as 66-03-00-N006);
DOTA-(β-Ala)-Sar₁₀-A-(SEQ ID NO: 3) (herein referred to as 66-03-00-N007);
Ac-(SEQ ID NO: 3)-A-Sar₆-K (herein referred to as 66-03-00-N008);
Ac-(SEQ ID NO: 3)-A-Sar₆-(PG) (herein referred to as 66-03-00-N009);
A-(SEQ ID NO: 7)-A (herein referred to as 66-03-03-N001);
A-(SEQ ID NO: 8)-A (herein referred to as 66-03-04-N001);
A-(SEQ ID NO: 10)-A (herein referred to as 66-03-06-N001);
A-(SEQ ID NO: 15)-A (herein referred to as 66-03-11-N001);
A-(SEQ ID NO: 22)-A (herein referred to as 66-03-28-N002);
A-(SEQ ID NO: 29)-A (herein referred to as 66-08-01-N001);
Ac-(SEQ ID NO: 29) (herein referred to as 66-08-01-N004);
Ac-(SEQ ID NO: 29)-A-Sar₆-K(DOTA) (herein referred to as 66-08-01-N016); and
Ac-A-(SEQ ID NO: 30)-A-Sar₆-K(Biot) (herein referred to as 66-08-01-N003), in particular:
the peptide ligand wherein the polypeptide has an amino acid sequence:
(β-Ala)-Sar₁₀-A-(SEQ ID NO: 3) (herein referred to as 66-03-00-N006).

16. The peptide ligand as defined in claim 1, wherein the pharmaceutically acceptable salt is selected from the free acid or the sodium, potassium, calcium, or ammonium salt.

17. The peptide ligand as defined in claim 1, wherein the CD38 is human CD38.

18. A drug conjugate comprising the peptide ligand as defined in claim 1, conjugated to one or more effector and/or functional groups.

19. The drug conjugate as defined in claim 18, wherein the one or more effector and/or functional groups are cytotoxic agents.

20. The drug conjugate as defined in claim 19, wherein said cytotoxic agent is maytansine.

21. The drug conjugate as defined in claim 19, wherein said cytotoxic agent is DM1 and the peptide ligand is (β-Ala)-Sar₁₀-A-(SEQ ID NO: 5) (herein referred to as 66-03-00-N006), and wherein the drug conjugate is of the formula:

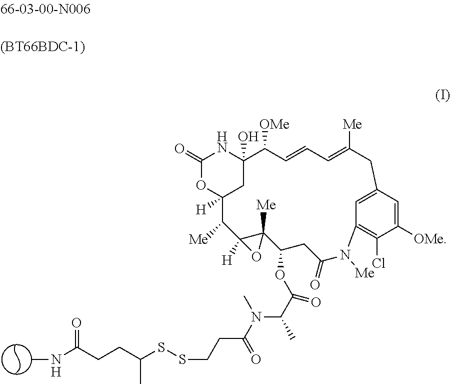 [SMILES: "(BT66BDC-1)" ; CNC(=O)CCC(C)SSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

22. A pharmaceutical composition which comprises the peptide ligand of claim 1, in combination with one or more pharmaceutically acceptable excipients.

23. A method for preventing, suppressing or treating a disease or disorder mediated by CD38 in a patient, comprising administering to the patient the peptide ligand as defined in claim 1.

24. A pharmaceutical composition which comprises the drug conjugate as defined in claim 18, in combination with one or more pharmaceutically acceptable excipients.

25. A method for preventing, suppressing or treating a disease or disorder mediated by CD38 in a patient, comprising administering to the patient the drug conjugate as defined in claim 18.
